(19) United States
(12) Patent Application Publication
(10) Pub. No.: US 2004/0191818 A1
(43) Pub. Date: Sep. 30, 2004

(54) Compositions and methods for diagnosing and treating autoimmune diseases

(76) Inventors: Margot Mary O'Toole, Newton, MA (US); Wei Liu, Sudbury, MA (US)

Correspondence Address: NIXON PEABODY, LLP, 401 9TH STREET, NW, SUITE 900, WASHINGTON, DC, 20004-2128, US

(21) Appl. No.: 10/786,720

(22) Filed: Feb. 26, 2004

Related U.S. Application Data
(60) Provisional application No. 60/449,753, filed on Feb. 26, 2003.
(60) Provisional application No. 60/449,693, filed on Feb. 26, 2003.
(60) Provisional application No. 60/449,795, filed on Feb. 26, 2003.

(51) Int. Cl. C12Q 1/68; A61K 38/17

(52) U.S. Cl. 435/6; 514/12

(57) ABSTRACT

Compositions and methods for diagnosing, preventing, or treating lupus nephritis (LN), systemic lupus erythematosus (SLE), or other autoimmune diseases. Lupus-related genes (LRGs) are identified in the present invention. These genes are differentially expressed in lupus-affected or lupus-predisposed tissues as compared to disease-free tissues. The genes and their expression products can be used as markers for diagnosing or monitoring SLE or LN. Modulators of the expression or protein activities of these genes can be used for the prevention or treatment of SLE/LN or other autoimmune diseases.

This application claims the benefit of U.S. Provisional Application Serial No. 60/449,693, filed Feb. 26, 2003 and entitled “Compositions and Methods for Diagnosing and Treating Autoimmune Disease,” U.S. Provisional Application Serial No. 60/449,753, filed Feb. 26, 2003 and entitled “Compositions and Methods for Diagnosing and Treating Autoimmune Disease,” and U.S. Provisional Application Serial No. 60/449,795, filed Feb. 26, 2003 and entitled “Compositions and Methods for Diagnosing and Treating Autoimmune Disease.”

This application incorporates by reference all materials recorded in compact discs labeled “Copy 1” and “Copy 2.” Each of the compact discs includes the file entitled “AM101331L Sequence Listing.ST25.txt” (3,406 KB, created on Feb. 26, 2004).

### TECHNICAL FIELD

The present invention relates to compositions and methods useful for the diagnosis, prevention, or treatment of lupus nephritis (LN), systemic lupus erythematosus (SLE), or other autoimmune diseases.

### BACKGROUND

Lupus nephritis (LN) is an inflammation of the kidney caused by systemic lupus erythematosus (SLE). SLE, commonly known as lupus, is an autoimmune rheumatic disease characterized by the deposition in tissues of autoantibodies and immune complexes leading to tissue injury. In contrast to autoimmune diseases such as multiple sclerosis and type 1 diabetes mellitus, SLE potentially involves multiple organ systems directly, and its clinical manifestations are diverse and variable. For example, some patients may demonstrate primarily skin rash and joint pain, show spontaneous remissions, and require little medication. At the other end of the spectrum are patients who demonstrate severe and progressive kidney involvement that requires immediate medical attention.

The serological hallmark of SLE, and the primary diagnostic test available until now, is elevated serum levels of IgG antibodies to constituents of the cell nucleus, such as double-stranded DNA (dsDNA), single-stranded DNA, and chromatin. Among these autoantibodies, IgG anti-dsDNA antibodies play a major role in the development of LN. LN is a serious condition in which the capillary walls of the kidney's blood purifying glomeruli are injured by the deposition of DNA/anti-DNA antibody complexes and the resulting complement activation and local inflammation. The disease is often chronic and progressive and may lead to eventual renal failure.

SLE is predominantly a female disease with an approximate female to male ratio of 9:1. In North America, it is estimated to affect 1 in 500 females between the ages of 20 to 40 years. It has been estimated that 45-75% of SLE patients eventually suffer kidney damage.

SLE shows a strong familial aggregation. While genetically-determined immune abnormalities are implicated in the cause of SLE, the triggering event is suggested to include both exogenous and endogenous factors, likely mutagenic in origin. Certain environmental and pharmacological agents, including UV light and drugs, such as procainamide and hydralazine, have been shown to trigger a lupus-like illness in genetically predisposed individuals.

Genetic studies of murine SLE have identified susceptibility loci in several inbred strains which spontaneously develop LN (for review, see Theofilopoulus, Immunol. Today, 15:150-58, 1995). These studies have included genome-wide searches for evidence of linkage using backcrosses or F₂ intercrosses of lupus mice such as MRL/LPR, NZB/NZW and NZM/Aeg2410 mice. Recent success in mapping a susceptibility locus for multiple sclerosis in the 5p14-p12 region, which is syngeneic to the murine locus Ea2, further supports the utility of this mouse-to-human approach. A genetic marker test for lupus has been generally described by Tsao et al. in U.S. Pat. No. 6,280,941.

MRL/MpJ-Fas^{lPr} mouse is a model for systemic lupus erythematosus-like autoimmune syndromes. The MRL/MpJ-Fas^{lPr} mice are generated by introducing a lymphoproliferation spontaneous mutation (Fas^{lPr}) within the fas gene into the MRL/MpJ mice. The fas protein is a cell surface antigen of about 35 kd that mediates apoptosis. It has a single transmembrane domain between its extracellular and cytoplasmic domains. The fas protein, a member of the tumor necrosis factor receptor superfamily, shows structural homology with several cell surface antigens, including the tumor necrosis factor and the low-affinity nerve growth factor receptor. The ligand for the fas protein, encoded by Fasl, is a member of the tumor necrosis factor family. Fas and its ligand are involved in down-regulating immune reactions.

MRL/MpJ-Fas^{lPr} mice show systemic autoimmunity, massive lymphadenopathy associated with proliferation of aberrant T cells, arthritis, and LN. Onset and severity of symptoms are dependent on genetic background, with the original MRL/MpJ background being most severely affected beginning about 8 weeks of age. The female and male mice die at an average age of 17 weeks and 22 weeks, respectively. It has been demonstrated that the Fas^{lPr} mutation is required for the development of LN and the subsequent death at an early age.

MRL/MpJ mice, the ancestral strain of MRL/MpJ-Fas^{lPr}, also exhibit autoimmune disorders but the symptoms are manifested much later in life compared to those of the MRL/MpJ-Fas^{lPr} mice. Starting at about three months of age, levels of circulating immune complexes rise greatly in the MRL/MpJ-Fas^{lPr} mouse but not in the wild-type control, MRL/MpJ. Also, beginning at 3 months MRL/MpJ-Fas^{lPr} mice exhibit very severe proliferative glomerulonephritis, whereas in the MRL/MpJ controls only mild glomerular lesions are usually detected. The MRL/MpJ wild-type females die at 73 weeks of age and males at 93 weeks, as in contrast to a lifespan of 17 weeks for females and 22 weeks for males in the MRL/MpJ mice homozygous for Fas^{lpr}. However, when the Fas^{lPr} mutation is bred into other strains (C57BL/6 for example), kidney function remains normal through life. It thus appears that the MRL/MpJ mice have inherited a predisposition to developing lupus which is accelerated in the presence of the Fas^{lPr} allele

Treatment for SLE is directed at controlling the symptoms with the hope of putting the disease into remission. There are several chemotherapeutic agents in commercial use and available for remedial purposes. Most of these agents are not without side effects, some of which are severe and debilitating to the patient. Some non-steroidal anti-inflammatory agents may cause stomach upset and changes in kidney function, which can mimic some lupus symptoms themselves. Some anti-malarial drugs, when required at high dosage levels over a prolonged time frame, may accumulate in the retina and cause loss of vision. Certain steroidal preparations are used for their anti-inflammatory activity. The steroids, however, can exhibit side effects such as pronounced swelling of the face and abdomen, weight gain, excessive growth of body hair, cataracts, osteoporosis and heart attacks. Use of immunosuppressants can also have serious side effects such as changes in bone marrow, increased risk of infection to which the body normally shows resistance and a slight increase in the risk of developing certain types of cancer.

Another method of treatment for SLE is to generate monoclonal antibodies against anti-DNA antibodies (i.e., anti-idiotypic antibodies) and then use these anti-idiotypic antibodies to remove the pathogenic anti-DNA antibodies from the patient's system (see U.S. Pat. No. 4,690,905, Diamond et al.). This approach, however, requires the removal of large quantities of blood for treatment in a process similar to hemodialysis. It is expensive and time-consuming, and is also associated with the risk of infection and/or hemorrhaging. Therefore, there remains a need for improved methods for diagnosing and treating SLE, as well as SLE-related diseases, such as LN.

### SUMMARY OF THE INVENTION

The present invention provides compositions and methods that are useful for the diagnosis, prevention, or treatment of LN, SLE, or other autoimmune diseases. Numerous lupus-related genes (LRGs) can be identified according to the present invention. These genes are differentially expressed in pre-symptomatic lupus-affected or -predisposed tissues as compared to disease-free tissues. In many embodiments, the LRGs of the present invention are also differentially expressed in early-stage lupus-affected tissues as compared to disease-free tissues. In many other embodiments, the different expression profiles of the LRGs in lupus-affected or lupus-predisposed tissues are not affected by age, gender, or Fas^{lPr} background. The LRGs of the present invention can be used as markers for diagnosing or monitoring SLE or LN. The LRGs can also be used as drug targets for the prevention or treatment of SLE, LN, or other autoimmune diseases.

In one aspect, the present invention provides methods useful for diagnosing or monitoring SLE or LN in a subject of interest. The methods include the steps of detecting an expression profile of at least one LRG gene in a biological sample of the subject, and comparing the expression profile to a reference expression profile of the LRG gene. In one embodiment, the LRG gene is over-expressed in both pre-symptomatic and early disease tissues as compared to disease-free tissues. In another embodiment, the LRG gene is under-expressed in both pre-symptomatic and early disease tissues.

The biological samples amenable to the present invention include, but are not limited to, urine samples, kidney samples, or other bodily fluid or tissue samples. In one embodiment, the biological samples are blood samples. Without limiting the present invention to any particular theory, the biological mechanism(s) involved in the up-regulation or down-regulation of LRGs in kidney tissues may also modulate the expression of the same genes in blood samples.

The expression profile of an LRG in a biological sample can be determined by using any method known in the art. Examples of these methods include, but are not limited to, RT-PCT, Northern Blot, in situ hybridization, slot-blotting, nuclease protection assay, nucleic acid arrays, or immunoassays. A variety of immunoassay formats are available for the present invention. They include, without limitation, latex or other particle agglutination, electrochemiluminescence, ELISAs, RIAs, sandwich or immunometric assays, time-resolved fluorescence, lateral flow assays, fluorescence polarization, flow cytometry, immunohistochemical assays, Western blots, and proteomic chips.

In many embodiments, the reference expression profile of an LRG and the expression profile being compared are determined using the same or comparable assays. In one example, the reference expression profile is an average expression profile of the LRG in disease-free tissues. In another embodiment, the reference expression profile is an average expression of the LRG in lupus-affected or lupus-predisposed tissues. The comparison between the expression profiles can be quantitative or qualitative. The comparison can be conducted based on absolute difference, expression ratio, or other measures that can represent a difference in expression profiles. In one example, pattern reorganization programs are used to compare expression profiles.

In one embodiment, the LRGs used in the present invention are selected from Table 1. In another embodiment, the LRGs are selected from Table 5b.

In another aspect, the present invention provides pharmaceutical compositions which include a pharmaceutically-acceptable carrier and at least one active component selected from the group consisting of (1) a polypeptide encoded by an LRG gene; (2) a variant of the polypeptide; and (3) a polynucleotide encoding the polypeptide or variant. In one embodiment, the LRG gene is over-expressed in lupus-affected or lupus-predisposed tissues as compared to disease-free tissues, and the pharmaceutical compositions are vaccine formulations capable of eliciting an immune response against lupus-affected or lupus-predisposed cells or components thereof. In one example, the lupus-affected or lupus-predisposed cells are human cells or tissues. In another example, the LRG gene is selected from Table 1. Any method known in the art may be used to administer the pharmaceutical compositions of the present invention into a subject to achieve the desirable therapeutic or prophylactic effect.

In yet another aspect, the present invention provides pharmaceutical compositions which include a pharmaceutically-acceptable carrier and at least one active component selected from the group consisting of (1) an agent capable of modulating the expression of an LRG gene; (2) an agent capable of binding to, or modulating a biological activity of, a polypeptide encoded by the LRG gene; and (3) a T cell activated by the polypeptide. The LRG gene can be either over-expressed or under-expressed in lupus-affected or lupus-predisposed tissues as compared to disease-free tissues. By administering the pharmaceutical compositions of the present invention or by contacting the compositions with lupus-affected or lupus-predisposed cells or tissues, the abnormality in the expression or activity of an LRG may be corrected or reduced, thereby ameliorating the syndrome or progression of SLE/LN.

In one embodiment, the LRGs are over-expressed in lupus-affected or lupus-predisposed tissues. The pharmaceutical compositions of the present invention include a polynucleotide which can inhibit the expression of the LRGs by RNAi or an anti-sense mechanism. In another embodiment, the pharmaceutical compositions of the present invention include antibodies or other molecules capable of binding to and inhibiting the biological activities of the LRG proteins.

In still another embodiment, the LRGs are under-expressed in lupus-affected or lupus-predisposed tissues. The pharmaceutical compositions of the present invention include agents that can stimulate the expression or protein activities of the LRGs. In a further embodiment, the pharmaceutical compositions of the present invention include gene therapy vectors which encode the LRGs or fragments thereof. Introducing the gene therapy vectors into a subject in need thereof may restore the expression or protein activies of the LRGs in lupus-affected or lupus-predisposed tissues.

The present invention also features diagnostic kits or assay systems that include probes for LRGs or their expression products. In one embodiment, the kits or systems include polynucleotide probes capable of hybridizing under stringent or highly stringent conditions to LRG transcripts, or the complements thereof. Examples of LRG transcripts include, but are not limited to, SEQ ID NOS: 1-29. In another embodiment, the kits or systems include antibodies or other polypeptide probes that can bind to LRG proteins. Examples of LRG proteins include, but are not limited to, SEQ ID NOS: 30-57.

In a further aspect, the present invention provides methods useful for identifying agents that are capable of modulating the expression or protein activities of LRG genes. The methods include the steps of contacting a candidate agent with lupus-affected or lupus-predisposed cells, and comparing expression profiles or protein activities of LRG genes in the cells before and after said contacting to determine if the agent can modulate the expression or protein activities of the LRG genes. The cells employed in these methods can be, without limitation, cell cultures or tissues cultures. In one example, the agent is administered into a subject (e.g., an animal model) to determine if the agent can modulate the expression profiles of LRGs in lupus-affected or lupus-predisposed cells in vivo.

In another aspect, the present invention provides methods useful for evaluating the effectiveness or efficacy of an agent in preventing or treating LN, SLE, or other autoimmune diseases. The methods include the steps of administering an agent to a lupus-affected or lupus-predisposed subject, and comparing expression profiles or protein activities of LRGs in biological samples of the subject before and after the administration to determine if the agent modulates the expression or protein activities of the LRG genes. Elimination or reduction of the abnormality in the expression or protein activities of the LRG genes is indicative of the effectiveness or efficacy of the agent.

Furthermore, the present invention provides host cells harboring transfected LRGs. These cells can be used for the treatment of SLE/LN. The present invention also provides knock-out animals in which the genomic sequence of at least one LRG is disrupted.

Other objects, features and advantages of the present invention will become apparent from the following detailed description. The detailed description and specific examples, while indicating preferred embodiments, are given for illustration only since various changes and modifications within the scope of the invention will become apparent to those skilled in the art from this detailed description. Further, the examples demonstrate the principle of the invention and should not be expected to specifically illustrate the application of this invention to all the examples of infections where it obviously will be useful to those skilled in the prior art.

### BRIEF DESCRIPTION OF THE DRAWINGS

The inventions of this application are better understood in conjunction with the following drawing. The drawing is provided for illustration, not limitation.

FIG. 1 is a flow chart describing steps for selecting lupus-related genes according to the present invention.

### DETAILED DESCRIPTION OF THE INVENTION

The present invention is directed to compositions and methods useful for the diagnosis, prevention, or treatment of SLE/LN or other autoimmune diseases, and to the identification of novel therapeutic agents for SLE/LN or other autoimmune diseases. The present invention is based on the discovery of lupus-related genes (LRGs) that are differentially expressed (e.g., over-expressed or under-expressed) in animals that are affected by or predisposed to SLE/LN as compared to SLE/LN-free animals. In many embodiments, the ratio of the average expression level of an LRG in SLE/LN-affected or SLE/LN-predisposed tissues over that in SLE/LN-free tissues is at least 1.5:1, 2:1, 3:1, 4:1, 5:1, or greater. In many other embodiments, the LRGs of the present invention are over-expressed in both pre-symptomatic and early-stage lupus-affected tissues. In still many other embodiments, the p-value of Student's t-test for the different expression profiles of an LRG in SLE/LN-affected or -predisposed tissues versus SLE/LN-free tissues is no greater than 0.01, 0.005, 0.0001, or lesser. LRGs down-regulated in SLE/LN-affected or SLE/LN-predisposed tissues are identified.

Various aspects of the invention are described in further detail in the following subsections. The use of subsections is not meant to limit the invention; subsections may apply to any aspect of the invention. In this application, the use of “or” means “and/or” unless stated otherwise.

LRGs and LN

In one embodiment, LRGs were identified by gene expression analysis using kidney RNA samples harvested from 4 different strains of mice, namely: MRL/MpJ-Fas^{lPr}, MRL/MpJ, C57BL6 and C57BL6/Fas^{lPr}. A gene analysis set of 5285 oligonucleotides was first selected using the criteria described in Examples. The expression frequency of each gene on these 5285 oligonucleotides in the gene analysis set was then determined for all C57BL6, C57BL6/Fas^{lPr}, MRL/MpJ-Fas^{lPr} and MRL/MpJ kidney samples (n=46).

Table 1 lists examples of the human orthologs of mouse LRG genes identified by the present invention. These genes include, but are not limited to, FSHD region gene1 (FRG1); glutamyl-prolyl-tRNA synthetase (EPRS); profilin 1 (PFN1); proteasome 26S subunit, non-ATPase, 8 (PSMD 8); axin 1 (AXIN1); guanine nucleotide binding protein, beta polypeptide 1 (GNB 1); collagen, type IV, alpha 3 (COL4A3); heat shock 10 kd protein 1 (chaperonin 10, HSPE1); dodecenoyl-Coenzyme A delta isomerase (DCI); recoverin (RCV1); secreted frizzled-related protein 1 (SFRPI); CD82 antigen (KAI1); and apolipoprotein M (APOM).

TABLE 1
Examples of Lupus-Related Genes (LRGs)

| | | cDNA Sequence | |
|---|---|---|---|
| | | (including isoforms or | Amino Acid |
| Gene Symbol | LocusID | alternative splicing) | Sequence |
| FRG1 | 2483 | SEQ ID NO: 1 | SEQ ID NO: 30 |
| EPRS | 2058 | SEQ ID NO: 2 | SEQ ID NO: 31 |
| PFN1 | 5216 | SEQ ID NO: 3 | SEQ ID NO: 32 |
| PSMD8 | 5714 | SEQ ID NO: 4 | SEQ ID NO: 33 |
| | | SEQ ID NO: 5 | |
| AXIN1 | 8312 | SEQ ID NO: 6 | SEQ ID NO: 34 |
| | | SEQ ID NO: 7 | SEQ ID NO: 35 |
| | | SEQ ID NO: 8 | SEQ ID NO: 36 |
| GNB1 | 2782 | SEQ ID NO: 9 | SEQ ID NO: 37 |
| COL4A3 | 1285 | SEQ ID NO: 10 | SEQ ID NO: 38 |
| | | SEQ ID NO: 11 | SEQ ID NO: 39 |
| HSPE1 | 3336 | SEQ ID NO: 12 | SEQ ID NO: 40 |
| DCI | 1632 | SEQ ID NO: 13 | SEQ ID NO: 41 |
| RCV1 | 5957 | SEQ ID NO: 14 | SEQ ID NO: 42 |
| SFRP1 | 6422 | SEQ ID NO: 15 | SEQ ID NO: 43 |
| APOM | 55937 | SEQ ID NO: 16 | SEQ ID NO: 44 |
| KAI1 | 3732 | SEQ ID NO: 17 | SEQ ID NO: 45 |
| FLJ22709 | 79629 | SEQ ID NO: 18 | SEQ ID NO: 46 |
| KIAA0063 | 9929 | SEQ ID NO: 19 | SEQ ID NO: 47 |
| LOC57019 | 57019 | SEQ ID NO: 20 | SEQ ID NO: 48 |
| TIM14 (homolog | 131118 | SEQ ID NO: 21 | SEQ ID NO: 49 |
| of yeast | | SEQ ID NO: 22 | SEQ ID NO: 50 |
| TIM14) | | SEQ ID NO: 23 | SEQ ID NO: 51 |
| | | SEQ ID NO: 24 | SEQ ID NO: 52 |
| GABRB3 | 2562 | SEQ ID NO: 25 | SEQ ID NO: 53 |
| | | SEQ ID NO: 26 | SEQ ID NO: 54 |
| FLJ30990 | 150737 | SEQ ID NO: 27 | SEQ ID NO: 55 |
| FLJ38991 | 285521 | SEQ ID NO: 28 | SEQ ID NO: 56 |
| CLN6 | 54982 | SEQ ID NO: 29 | SEQ ID NO: 57 |

### The Biochemical and Biological Characteristics of the LRGs

1. FRG1 (FSHD Region Gene 1)

FRG1 (FSHD region gene 1) was identified as a gene related to facioscapulohumeral muscular dystrophy (FSHD), an autosomal dominant neuromuscular disorder. The disease is characterized by the weakness of the muscles of the face, upper-arm and shoulder girdle. The FRG1 gene has been mapped to chromosome locus 4q35 and is closely linked to D4F1O4S1. This evolutionarily conserved gene belongs to a multi-gene family with FRG1 related sequences on multiple chromosomes. The mature chromosome 4 FRG1 transcript is 1042 bp in length and contains nine exons which encode a putative protein of 258 amino acid residues. The biological function of the FRG1 protein and its role in the pathophysiology of FSHD still remain to be elucidated.

2. Glutamyl-prolyl-tRNA Synthetase (EPRS)

AminoacyltRNA synthetases are a class of enzymes that charge tRNAs with their cognate amino acids. In humans, the glutamyl-tRNA synthetase (GluRS) and prolyl-tRNA synthetase (ProRS) activities are contained within a single polypeptide chain, even though these enzymes belong to different classes and are thought to have evolved along independent evolutionary pathways. Glutamyl-prolyl-tRNA synthetase is made up of 1,440 amino acids encoded by 29 exonx. The exons encoding the glutamyl-specific and prolyl-specific parts of the enzyme are clustered at opposite ends of the gene, separated by a long intervening DNA section with a number of exons which encode functions that may be involved in the organization of the mammalian multienzyme synthetase complex.

3. Profilin 1 (PFNJ)

The protein encoded by this gene is a ubiquitous action monomer-binding protein belonging to the profilin family. It is thought to regulate actin polymerization in response to extracellular signals. Deletion of this gene is associated with Miller-Dieker syndrome, a developmental defect of the brain caused by incomplete neuronal migration Profilm 2, another member of the profilin family, has been identified as a endotbelial cell auto antigens in SLE (Frampton et al Rheumatology, 39:1114-1120, 2000).

4. Proteasome 26S Subunit, non-A TPase 8 (PSMD8)

PSMD8 is one of the subunits in 26S proteasome, a protein complex involved in the degradation of cellular proteins through the ubiquitin/proteasome pathway. Generally, the ubiquitin/proteasome pathway involves two successive steps: 1) conjugation of multiple ubiquitin moieties to the substrate and 2) degradation of the tagged protein by the downstream 26S proteasome complex. Proteasome is a dynamic protein complex forming multiple interactions with transiently associated subunits and cellular factors that are necessary for functions such as cellular localization, presentation of substrates, substrate-specific interactions, or generation of various products.

5. Axin 1 (AXIN1)

Axin 1 is a component of the Wnt signaling pathway and negatively regulates this pathway. The Wnt signaling pathway is conserved in various species from worms to mammals, and plays important roles in development, cellular proliferation, and differentiation. Wnt stabilizes cytoplasmic beta-catenin, which stimulates the expression of genes including c-myc, c-jun, fra-1, and cyclin D1. Other components of the Wnt signaling pathway, including Dv1, glycogen synthase kinase-3beta, beta-catenin, and adenomatous polyposis coli, interact with Axin, and the phosphorylation and stability of beta-catenin are regulated in the Axin complex. Thus, Axin acts as a scaffold protein in the Wnt signaling pathway, thereby regulating cellular functions. Human Axin is strongly similar to murine Axin and may also regulate embryonic axis formation.

6. Guanine Nucleo Tide Binding Protein, Beta Polypeptide 1 (GNB1)

Heterotrimeric guanine nucleotide-binding proteins (G proteins), which integrate signals between receptors and effector proteins, are composed of an alpha, a beta, and a gamma subunit. These subunits are encoded by families of related genes. The GNB1 gene encodes a beta subunit. Beta subunits are important regulators of alpha subunits, as well as of certain signal transduction receptors and effectors. This gene uses alternative polyadenylation signals.

7. Collagen, Type IV, Alpha 3 (COL4A3)

This gene encodes one of the six subunits of type IV collagen, the major structural component of basernent membranes. It plays a role in Goodpasture syndrome, a rare autoimmune disease that leads to autoimmune attack to lungs and kidneys. In the Goodpasture syndrome, autoantibodies bind to the collagen molecules in the basement membranes of alveoli and glomeruli. The epitopes that elicit these autoantibodies are localized largely to the non-collagenous C-terminal domain of the protein A specific kinase phosphorylates ammo acids in this same C-terminal region and the expression of this kinase is up-regulated during pathogenesis. There are six alternate transcripts that appear to be unique to this human subunit gene and alternate splicing is restricted to the six exons that encode this domain.

COL4A3 gene is also linked to an autosomal recessive form of Alport syndrome. Alport syndrome, affecting about one in 5,000 persons, is hereditary glomerulonephritis that is caused by mutation of one or the other of several COL4A genes that specify alpha chains of basement membrane (Type IV) collagen, or by mutation of unknown genes. Especially in males, the resultant chronic nephritis progresses via uremic syndrome to end-stage renal disease treatable only by dialysis or by kidney transplantation. In various families, nephritis may be associated with various combinations of hearing loss, lenticonus and other eye disorders, immunologic abnormality of skin, disorders of platelets, abnormalities of white blood cells, or smooth muscle tumors.

8. Heat Shock 10 kd Protein 1 (Chaperonin 10, HSFEJ)

Chaperonins are a subclass of molecular chaperones that assist both the folding of newly synthesized proteins and the maintenance of proteins in a folded state during periods of stress.

Chaperonin 10 interacts with chaperonin 60 (HSPD1) to retold denatured proteins. Human HSPE1 shares very high homology to murine Hspe1.

9. Dodecenoyl-Coenzyme A Delta Isomerase (DCI)

Cellular energy metabolism is largely sustained by mitochondrial beta-oxidation of saturated and unsaturated fatty acids. DCI is the link in mitochondrial beta-oxidation of unsaturated and saturated fatty acids and is essential for the complete degradation of the fatty acids and for maximal energy yield. It catalyzes the transformation of 3-cis and 3-trans intermediates arising during the stepwise degradation of all cis-, mono-, and polyunsaturated fatty acids to the 2-trans-enoyl-CoA intermediates. Mitochondrial beta-oxidation of unsaturated fatty acids is interrupped in DCI (−/−) mice at the level of their respective 3-cis- or 3-trans-enoyl-CoA intermediates. Fasting DCI (−/−) mice accumulate unsaturated fatty acyl groups in ester lipids and deposit large amounts of triglycerides in hepatocytes (steatosis). The entire human DCI gene encompases approximately 12.5 kb, and the coding sequence is distributed over seven exons. The human DCI gene locus was assigned to chromosome 16 by use of human-rodent somatic cell hybrids and to chromosome 16p13.3 by chromosomal in situ suppression hybridization studies.

10. Recoverin (RCV1)

Recoverin is a member of the EF-hand superfamily. It is normally expressed only in the retina and serves as a calcium sensor in retinal rod cells. A myristoyl or related fatty acyl group covalently attached to the N-terminus of recoverin facilitates the binding of recoverin to retinal disk membranes by a mechanism known as the Ca²⁺-myristoyl switch.

Aberrant expression of recoverin, however, has been observed in several cancer tissues and may cause a very rare autoimmune disease, cancer-associated retinopathy (CAR), the etiology of which is not yet clear. Autoantibodies against recoverin have been found in CAR patients with a few kinds of cancer (endothelial carcinoma, breast cancer, epithelial ovarian carcinoma, and lung cancer). As for lung cancer, the majority of CAR cases mediated by anti-recoverin autoantibodies have been revealed in patients with the most malignant lung cancer, small cell lung carcinoma (SCLC), and only one similar case has been described for a patient with non-small lung carcinoma (Bazhin et al., Lung Cancer, 34:99-104, 2001). The common feature of all these anti-recoverin-positive patients, irrespective of the type of cancer, is the presence of both the CAR syndrome and high titers (>1:1,000) of the underlying autoantibodies in their serum.

Recoverin-specific CTLs in the peripheral blood of CAR patients recognize recoverin-expressing tumor cells. An experimental mouse model has been generated to test the induction of recoverin-specific anti-tumor CTL, and to analyze retinal function using electroretinogram (ERG) (Maeda et al., Eur. J. Immunol., 32:2300-2307, 2002). It was found that a peptide, R64 (AYAQHVFRSF), derived from recoverin that induces anti-tumor CTL in humans, produced a recoverin-specific CTL response in Balb/c mice and significant growth inhibition of recoverin-expressing syngeneic MethA fibrosarcoma cells in vivo. Furthermore, elevated anti-recoverin antibodies correlated with decreased ERG amplitudes in recoverin-, recoverin-expressing-tumor- and R64-treated mice. These data suggest that recoverin contains amino acid sequences that may not only cause retinal dysfunction, but also induce anti-tumor CTL and tumor regression.

Anti-recoverin antibodies are also found to be present in patients with retinitis pigmentosa (RP). Since 40% of patients with RP have no family history, it has been suggested that some patients may have an underlying autoimmune process causing or contributing to their retinopathy. A study screening serum samples from 521 patients diagnosed with RP found anti-recoverin immunoreactivity in 10 patients without systemic malignancy but with clinical findings consistent with RP (Heckenlively, Arch. Ophthalmol., 11 8:1525-33, 2000). This result suggests that there are other immunogenic mechanisms occurring in the formation of anti-recoverin antibodies in addition to the putative tumor-mediated mechanisms. The close connection between recoverin and autoimmune diseases makes recoverin a strong candidate for lupus markers.

11. Secreted Frizzled-Related Protein 1 (SFRP1)

SFRP is a newly discovered family of secreted glycoproteins that function to modulate signaling activity of Wnt, a family of highly conserved secreted signaling molecules that regulate cell-to-cell interactions during embryogenesis. SFRP proteins share sequence homology with the extracellular domain of the Wnt receptor (frizzled) and are capable of binding to Wnt. Thus, SFRPs function to antagonize Wnt activity by sequestering Wnt and preventing its binding to the frizzled receptor.

SFRP1 contains an N-terminal domain homologous to the putative Wnt-binding site of Frizzled (Fz domain) and a C-terminal heparin-binding domain with weak homology to netrin. Both domains are cysteine-rich, having 10 and 6 cysteines in the Fz and heparin-binding domains, respectively.

SFRP1 plays an important rule in metanephric kidney development and functions as a modulator of Wnt signaling (Yoshino et al., Mech. Dev., 192:45-55, 2001). SFRP1 is distributed throughout the medullary and cortical stroma in the metanephros, but is absent from condensed mesenchyme and primitive tubular epithelia of the developing nephron where Wnt-4 is highly expressed. In cultures of isolated, induced rat metanephric mesenchymes, SFRP1 blocked events associated with epithelial conversion (tubulogenesis and expression of lim-1, SFRP2 and E-cadherin); however, it had no demonstrable effect on early events (compaction of mesenchyme and expression of wt1). SFRP1 binds Wnt-4 with considerable avidity and inhibits the DNA-binding activity of TCF, an effector of Wnt signaling.

Wnt family of embryonic differentiation genes also modulate growth of malignant glioma cells in vitro and in vivo and inhibit cellular migration in vitro (Roth et al., Oncogene, 19:4210-4220, 2000). It was found that SFRPs promote survival under non-supportive conditions and inhibit the migration of glioma cells. It was also suggested that the regulation of these cellular processes involves expression of MMP-2 and tyrosine phosphorylation of beta-catenin. These data support a function for Wnt signaling and its modulation by SFRPs in the biology of human gliomas.

SFRP1's involvement in metanephric kidney development and the Wnt signaling pathway and its association with predisposition to LN in the MRL/MpJ mice, support a claim of the possible involvement of SFRP1 in the development of LN.

12. Apolipoprotein M (APOM)

APOM is a recently discovered protein. It is a 26-kDa protein present in a protein extract of triglyceride-rich lipoproteins (TGRLP). The isolated APOM cDNA (734 base pairs) encoded a 188-amino acid residue-long protein. The mRNA of APOM was detected in the liver and kidney. Western blotting demonstrated APOM to be present in high density lipoprotein (HDL) and to a lesser extent in TGRLP and low density lipoproteins (LDL). The first 20 amino acid residues of APOM constituted a hydrophobic segment with characteristic features of a signal peptide. These amino acid residues, however, are retained in the mature protein because of the lack of a signal peptidase cleavage site. In vitro translation in the presence of microsomes demonstrated translocation of APOM over the membrane and glycosylation (Xu et al., J. Biol. Chem., 274:31286-31290, 1999).

Sensitive sequence searches, threading and comparative model building experiments revealed that APOM is structurally related to the lipocalin protein family. In a 3D model, characterized by an eight-stranded anti-parallel beta-barrel, a segment including Asn135 could adopt a closed or open conformation. Asn135 in wild-type APOM is glycosylated, suggesting that the segment is solvent exposed. APOM also displays two strong acidic patches of potential functional importance, one around the N-terminus and the other next to the opening of the beta-barrel (Duan, FEBS Lett., 499:127-132, 2001). It was found that platelet-activating factor (PAF) significantly enhanced the APOM mRNA levels and the secretion of APOM in HepG2 cell cultures (Xu et al., Biochem. Biophys. Res. Commun., 292:944-950, 2002). However, tumor necrosis factor alpha (TNF alpha) and interleukin-1alpha (IL-1alpha) had no effect on APOM expression in HepG2 cells. Furthermore, Lexipafant, a PAF-receptor (PAF-R) antagonist significantly suppressed the mRNA level and the secretion of APOM in HepG2 cells in a dose-dependent manner. Neither PAF nor Lexipafant influenced the mRNA levels and the secretion of APOA-1, APOB and APOE in HepG2 cells, indicating that the effects of PAF or Lexipafant on the APOM production in hepatic cells are selective for APOM.

The human APOM gene is located in the major histocompatibility complex class II region on chromosome 6. This region codes for a large number of genes crucial to pro-inflammatory response function. Therefore, despite the lack of effect of pro-inflammatory mediators TNF alpha and IL-1 alpha on APOM expression, APOM may be involved in pro- inflammatory processes. A role for APOM in such processes would be consistent with a role in the processes leading to tissue destruction seen in LN.

13. KAI1/CD82

KAI1/CD82 is a member of the transmembrane 4 superfamily (TM4SF). It is a multifunctional molecule that is involved in activation, costimulation, and cell spreading of T cells. Studies have shown that KAI1/CD82 associates with CD4 or CD8 and delivers costimulatory signals for the TCR/CD3 pathway. Costimulation through both CD82 and CD3 induced up-regulation of both IL-2 and IFN-gamma mRNA synthesis (but not of IL-4) and an increased expression of HLA class I molecules at the cell surface, which was inhibited by anti-IFN-gamma Ab (Lebel-Binay et al., J. Immunol., 155:101-110, 1995).

It was found by sequential immunoprecipitation analysis that KAI1/CD82 is associated with HLA class I heavy chain in various B cell lines (Lagaudriere-Gesbert et al., J. Immunol., 158:2790-2797, 1997). Cocapping experiments confirmed the molecular association of CD82 and HLA class I at the cell surface of these B cell lines. These results suggest that association of CD82-MHC-I may interfere with the capacity of the MHC class I complex to protect targets from NK-mediated cytotoxicity. KAI1/CD82 is also a resident of MHC class II compartments where it associates with HLA-DR, -DM, and -DO molecules and may play an important role in the late stages of MHC class II maturation (Hammond et al., J. Immunol., 161:3282-91, 1998).

Northern blot analysis showed quite variable expression of the mouse CD82 gene among different organs. The highest expression was seen in the spleen and the kidney. The expression was low in skeletal muscle and hardly detectable in the heart.

Recently, it was found that KAI1/CD82 engagement leads to the tyrosine phosphorylation and association of both the Rho GTPases guanosine exchange factor Vav1 and adapter protein SLP76, suggesting that Rho GTPases participate in KAI1/CD82 signaling. There is also evidence for distinctive signaling of CD82- and betal integrin-mediated costimulation at the transcriptional level of IL-2 gene in human T cells. While lymphocytic infiltration and activation have long been appreciated as hallmarks of lupus nephritis, the higher than normal levels of KAI1/CD82 in kidneys in the pre-symptomatic state suggest a role for this molecule early in the disease pathway.

It should be noted that KAI1/CD82 has been identified as a prostate cancer suppressor gene. Down-regulation of KAI1/CD82 has been reported in a variety of malignancies, such as cervical carcinoma and ovarian cancer. It was also reported that over-expression of KAI1/CD82 suppresses in vivo metastasis in breast cancer cells. An analysis of tumor tissues from 151 lung cancer patients indicated that the overall survival rate of patients with KAI1/CD82-positive tumors was significantly higher than that of patients with KAI1/CD82-negative tumors, and that the overall survival rate of patients with KAI1/CD82-positive adenocarcinoma was also much higher than that of individuals whose adenocarcinoma had reduced KAI1/CD82 expression (Adachi, Cancer Res., 56:1751-1755, 1996). Multivariate analysis with the Cox regression model indicated that KAI1/CD82 positivity correlated best with the overall survival rate, except for lymph node status. These data suggest that high KAI1/CD82 gene expression by tumors of the lung may be associated with a good prognosis.

14. FLJ22709

FLJ22709 encodes a hypothetical protein. The gene has LocusID 79629 and is reported to have cytogenetic location 19p13.12.

15. KIAA0063

KIAA0063 has LocusID 9929 and is reported to have cytogenetic location 22ql3.1.

16. LOC57019

LOC57019 encodes a hypothetical protein. The gene has LocusID 57019 and is reported to have cytogenetic location 16q13-q21.

17. TIM14 (Homolog of Yeast TIM14)

The gene is similar to RIKEN cDNA 1810055D05. It has LocusID 131118 and is reported to have cytogenetic location 3q27.2.

18. GABRB3

GABRB3 has LocusID 2562 and is reported to have cytogenetic location 15q11.2-q12. GABRB3 encodes gamma-aminobutyric acid (GABA) A receptor, beta 3. The gamma-aminobutyric acid (GABA) A receptor is a multisubunit chloride channel that mediates the fastest inhibitory synaptic transmission in the central nervous system. Alternative splicing generates at least two transcript variants. Deletion mutation of this gene may be involved in the pathogenesis of Angelman syndrome and Prader-Willi syndrome.

19. FLJ30990

FLJ30990 encodes a hypothetical protein which has LocusID150737 and reported cytogenetic location 2q31.3.

20. FLJ38991

FLJ38991 encodes a hypothetical protein which has LocusID 285521 and reported cytogenetic location 4q21.1.

21. CLN6

CLN6 encodes ceroid-lipofuscinosis, neuronal 6, late infantile, variant. The gene is also known as FLJ20561 which has LocusID 54982 and reported cytogenetic location 15q22.31.

The biochemical and biological characteristics of the LRGs with known functions further support their involvement in the development or progression of auto-immune diseases such as SLE/LN. The current understanding on LRGs' structures (including secondary structures) or functions provides a basis for clinical applications of LRGs in the diagnosis, prevention, or treatment of SLE/LN.

### LRGs as Markers for SLE and LN

The LRGs of the present invention, or the polypeptides and polypeptides encoded thereby (hereinafter referred to as LRPNs and LRPPs, respectively), can be used as markers for diagnosing or monitoring SLE/LN. These markers may be components in the disease mechanism and therefore can be used as therapeutic targets for the treatment and prevention of SLE/LN. While mouse models were used for the initial differentiation expression analysis, it is well appreciated in the art that a dysfunctional gene that leads to disease in animals can also, when dysfunctional, lead to a similar syndrome in humans. The present invention encompasses human LRGs. In addition, LRGs in other organisms can also be identified and used for the study of SLE/LN or for the identification of drugs that are useful for preventing or treating SLE/LN.

Lupus is a complex disease whose clinical manifestations are diverse and variable. Patients vary with respect to both disease course and clinical response, and these variations probably reflect differences in type of lupus disease present. The LRGs of the present invention can be used to provide more precise and specific diagnoses, thereby leading to more effective therapy choices.

Polynucleotide, polypeptide, or other types of probes for the LRGs of the present invention can be prepared using a variety of methods. These probes can be used individually or coupled to carriers. In one example, the LRG probes are arrayed on solid supports (e.g., biochips) to detect LRG mRNA or proteins. In another example, anti-LRG antibodies are developed using conventional means. The probes of the present invention can be used to provide diagnosis or prognosis information for a subject of interest or to assess the efficacy of a treatment or therapy of SLE/LN. Comparison of expression levels of LRGs at different stages of the disease progression may also provide means for long-term prognosis, including survival. In addition, LRG polymorphism may be indicative of a subject's susceptibility to SLE/LN.

LRGs (including LRG promoters or other regulatory sequences) and LRG gene products can be targets for therapeutic or prophylactic agents. They can also be used to generate gene therapy vectors to inhibit SLE/LN.

Without limitation as to mechanism, the invention is based in part on the principle that modulation of LRG expression may ameliorate SLE/LN. The modulation may occur at transcriptional, post-transcriptional, translational, and post-translational levels. For example, an LRG promoter may be targeted to inhibit transcription. An LRG mRNA may be targeted by antisense molecules to prevent translation. The post-translational processing of an LRG protein, such as leader peptide removal, glycosylation and dimerization, may also be targeted.

The discovery of the LRG expression patterns in SLE/LN-affected animals allows for the screening of agents that can modulates LRG expression or LRG activity. The agents may be screened by their effects on LRG expression at the mRNA or protein level, or by their effect on the activity of the LRG product.

In one embodiment, a modulator of LRG expression or LRG activity may be used as a therapeutic agent for SLE or LN. The modulator may be a polynucleotide (such as an antisense oligonucleotide or an RNAi sequence), a polypeptide (such as an anti-LRG antibody), an LRG mutant having a dominant negative effect on an activity of the wild-type LRG, a viral or non-viral gene therapy vector, or any other small molecule or biomolecule that is capable of inhibiting LRG activity or LRG expression. The formulation of such a modulator into pharmaceutical compositions is described below.

### Isolated LRG Polynucleotides

One aspect of the invention pertains to isolated polynucleotide fragments sufficient for use as hybridization probes to identify LRG products in a sample, as well as nucleotide fragments for use as PCR primers of the amplification or mutation of the nucleic acid molecules which encode an LRPP of the present invention. Another aspect of the invention pertains to isolated polynucleotides that encode an LRPP, or a fragment or mutant thereof.

A polynucleotide molecule comprising an LRPP, or a homolog, fragment or variant thereof, can be isolated using standard molecular biology techniques. An LRG polynucleotide variant includes polynucleotides that are capable of hybridizing to the original polynucleotide, or the complement thereof, under reduced stringent conditions. In many embodiments, a variant can hybridize to the original polynucleotide, or the complement thereof, under stringent conditions or highly stringent conditions. Examples of conditions of different stringency are listed in Table 2. Highly stringent conditions are those that are at least as stringent as conditions A-F; stringent conditions are at least as stringent as conditions G-L; and reduced stringency conditions are at least as stringent as conditions M-R. As used in Table 2, hybridization is carried out under a given hybridization condition for about 2 hours, followed by two 15-minute washes under the corresponding washing condition(s).

TABLE 2
Stringency Conditions

| Stringency | Poly-nucleotide | Hybrid | Hybridization | Wash Temp. |
|---|---|---|---|---|
| Condition | Hybrid | Length (bp)¹ | Temperature and Buffer^{H} | And Buffer^{H} |
| A | DNA:DNA | >50 | 65° C.; 1xSSC -or- | 65° C.; 0.3xSSC |
| | | | 42°; 1xSSC, 50% formamide | |
| B | DNA:DNA | >50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | >50 | Tₚ*; 1SSX | Tₚ*; 1SSX |
| E | RNA:RNA | >50 | 70° C.; 1xSSC -or- | 70° C.; 0.3xSSC |
| | | | 50° C.; 1xSSC, 50% formamide | |
| F | RNA:RNA | >50 | T_{F}*; 1xSSC | T_{F}*; 1xSSC |
| G | DAN:DNA | >50 | 65° C.; 4xSSC -or- | 65° C.; 1xSSC |
| | | | 42° C.; 4xSSC, 50% formamide | |
| H | DNA:DNA | >50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC; 50% formamide | |
| J | DNA:RNA | >50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | >50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | >50 | T_{N}*; 6xSSC | T_{N}*; 6xSSC |
| O | DNA:RNA | >50 | 55° C.; 4xSSC -or- | 55° C.; 4xSSC |
| | | | 42° C.; 6xSSC, 50% formamide | |
| P | DNA:RNA | >50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | >50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequences are hybridized, the hybrid length can be determined by aligning the sequences of the polynucleotides and identifying the region or regions of optimal sequence complementarity. | | | | |
| ^{H}SSPE (1xSSPE is 0.15 M NaCl, 10 mM NaH₂PO₄, and 1.25 mM EDTA, pH 7.4) can be substituted for SSC (1xSSC is O.15 M NaCl and 15 mM sodium citrate) in the hybridization and wash buffers. | | | | |
| T_{B}* -T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + bases). | | | | |
| # For hybrids between 18 and 49 base pairs in length, Tₘ(° C.) = 8 1.5 + 16.6(log₁₀Na⁺) + 0.41 (% G + C) − (600/N), where N is the number of bases in the hybrid, and Na⁺ is the molar concentration of sodium ions in the hybridization buffer (Na⁺ for 1xSSC = 0.165 M). | | | | |

An LRG polynucleotide variant of the present invention may differ from its original LRG polynucleotide by one or more substitutions, additions, and/or deletions. For instance, an LRG polynucleotide variant can have 1, 2, 5, 10, 15, 20, 25 or more nucleotide substitutions, additions or deletions. In one embodiment, the modification(s) is in-frame such that the modified polynucleotide can be transcribed and translated to the original or intended stop codon. In another embodiment, the biological activity is reduced/enhanced by less than 50%, 40%, 30%, 20%, 10% or lesser as compared to the original activity.

Probes or primers for detecting or amplifying LRGs or their transcripts can be DNA, RNA, PNA, or other forms of polynucleotides. The probes or primers can have any desirable length. For instance, the probes/primers can have at least about 7, 15, 25, 50, 75, 100, 125, 150, 175, 200, 225, 250, 275, 300, 325, 350, 400 or more consecutive nucleotides. of an LRG, or a polynucleotide transcribed thereof. In many embodiments, the probes or primers can hybridize under stringent conditions to the respective LRG transcripts, or the complements thereof.

In one embodiment, LRG probes comprise label groups. The label groups can be, for example, a radioisotope, a fluorescent compound, an enzyme, or an enzyme co-factor. Such probes can be used as a part of a diagnostic or test kit for identifying cells or tissue in which an LRG is differentially expressed (e.g., over- or under-expressed), or in which greater or fewer copies of an LRG exist.

The invention also encompasses homologs of the LRGs of other species. Gene homologs are well understood in the art and are identifiable from databases such as the Pubmed-Entrez database.

In addition, the invention encompasses polynucleotide molecules which are structurally different from the original molecules but substantially retain their original functions or other properties. Such molecules include allelic variants as described below.

In addition to the nucleotide sequences of the LRGs, it will be appreciated by those skilled in the art that DNA sequence polymorphisms that lead to changes in the amino acid sequences of the proteins encoded by the LRGs may exist within a population (e.g., the human population). Such genetic polymorphisms in the LRGs may exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation). As used herein, the phrase “allelic variant” includes a nucleotide sequence which occurs at a given locus and a polypeptide encoded by the nucleotide sequence.

In addition to naturally-occurring allelic variants of an LRG that may exist in the population, the skilled artisan will further appreciate that changes can be introduced by mutation into the nucleotide sequences of the LRG, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) may be predicted to be unamenable to alteration.

Accordingly, another aspect of the invention pertains to polynucleotides encoding the LRG proteins that contain changes in amino acid residues that are not essential for activity. Such proteins differ in amino acid sequence from the original LRG protein encoded by the LRG, yet retain biological activity. In one embodiment, the protein comprises an amino acid sequence that has at least about 60%, 65%, 70%, 75%, 80%, 85%, 90%, 95%, 98% or more sequence identity or similarity to an LRG protein.

In yet other aspect of the invention, the polynucleotides of the LRGs may comprise one or more mutations. An isolated polynucleotide molecule encoding a protein with a mutation can be created by introducing one or more nucleotide substitutions, additions or deletions into the nucleotide sequence of the polynucleotide, such that one or more amino acid substitutions, additions or deletions are introduced into the encoded protein. Such techniques are well-known in the art. Mutations can be introduced into an LRG by standard techniques, such as site-directed mutagenesis and PCR-mediated mutagenesis. In many cases, conservative amino acid substitutions can be made at one or more predicted non-essential amino acid residues. Alternatively, mutations can be introduced randomly along all or part of a coding sequence of the LRG or cDNA, such as by saturation mutagenesis, and the resultant mutants can be screened for biological activity to identify mutants that retain activity. Following mutagenesis, the encoded protein can be expressed recombinantly and the activity of the protein can be determined.

In yet another aspect of the invention, a polynucleotide may encode an LRPP containing mutations in amino acid residues which result in inhibition of LRPP activity after dimerization with a wild-type LRPP. These mutated LRPPs may be used to inhibit LRPP activity in an SLE/LN patient.

A polynucleotide may be further modified to increase stability in vivo. Possible modifications include, but are not limited to, the addition of flanking sequences at the 5′ and/or 3′ ends; the use of phosphorothioate or 2-o-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Another aspect of the invention pertains to isolated polynucleotide molecules, which are antisense to an LRG. An “antisense” polynucleotide comprises a nucleotide sequence which is complementary to a “sense” polynucleotide that encodes a protein, e.g., complementary to the coding strand of a double-stranded cDNA molecule or complementary to an mRNA sequence. Accordingly, an antisense polynucleotide can form hydrogen bonds with a sense polynucleotide. The antisense polynucleotide can be complementary to an entire coding strand of a gene of the invention or to only a portion thereof. In one embodiment, an antisense polynucleotide molecule is antisense to a “coding region” of the coding strand of a nucleotide sequence of the invention. The term “coding region” includes the region of the nucleotide sequence comprising codons which are translated into amino acids. In another embodiment, the antisense polynucleotide molecule is antisense to a “noncoding region” of the coding strand of a nucleotide sequence of the invention.

Antisense polynucleotides of the invention can be designed according to the rules of Watson and Crick base pairing. The antisense polynucleotide molecule can be complementary to the entire coding region of an mRNA corresponding to a gene of the invention, but it can also be an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense polynucleotide (e.g., an antisense oligonucleotide) can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenosine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. Alternatively, the antisense polynucleotide can be produced biologically using an expression vector into which a polynucleotide has been subcloned in an antisense orientation.

The antisense polynucleotide molecules of the invention are typically administered to a subject or generated in situ such that they hybridize with or bind to cellular mRNA and/or genomic DNA encoding an LRG to thereby inhibit expression of the protein, e.g., by inhibiting transcription and/or translation. The hybridization can be by conventional nucleotide complementarity to form a stable duplex, or, for example, in the cases of an antisense polynucleotide molecule which binds to DNA duplexes, through specific interactions in the major groove of the double helix. An example of a route of administration of antisense polynucleotide molecules of the invention includes direct injection at a tissue site (e.g., intestine or blood). Alternatively, antisense polynucleotide molecules can be modified to target selected cells and then administered systemically. For example, for systemic administration, antisense molecules can be modified such that they specifically bind to receptors or antigens expressed on a selected cell surface, e.g., by linking the antisense polynucleotide molecules to peptides or antibodies which bind to cell surface receptors or antigens. The antisense polynucleotide molecules can also be delivered to cells using the vectors described herein. To achieve sufficient intra-cellular concentrations of the antisense molecules, vector constructs in which the antisense polynucleotide molecule is placed under the control of a strong promoter can be used.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2-o-methylribonucleotide or a chimeric RNA-DNA analogue.

In still another embodiment, an antisense polynucleotide is a ribozyme. Ribozymes are catalytic RNA molecules with ribonuclease activity which are capable of cleaving a single-stranded polynucleotide, such as an mRNA, to which they have a complementary region. Thus, ribozymes can be used to catalytically cleave mRNA transcripts of an LRG to thereby inhibit translation of said mRNA. A ribozyme having specificity for an LRG can be designed based upon the nucleotide sequence of the LRG. An mRNA transcribed from an LRG can be used to select a catalytic RNA having a specific ribonuclease activity from a pool of RNA molecules. Alternatively, expression of an LRG can be inhibited by targeting nucleotide sequences complementary to the regulatory region of these genes (e.g., the promoter and/or enhancers) to form triple helical structures that prevent transcription of the gene in target cells.

Expression of an LRG can also be inhibited using RNA interference (RNAᵢ). This is a technique for post-transcriptional gene silencing (“PTGS”), in which target gene activity is specifically abolished with cognate double-stranded RNA (“dsRNA”). In many embodiments, dsRNA of about 21 nucleotides, homologous to the target gene, is introduced into the cell and a sequence specific reduction in gene activity is observed. RNA interference provides a mechanism of gene silencing at the mRNA level. RNAi offers an efficient and broadly applicable approach for gene knock-out as well as for therapeutic purposes.

Sequences capable of inhibiting gene expression by RNA interference can have any desired length. For instance, the sequence can have at least 15, 20, 25, or more consecutive nucleotides. The sequence can be dsRNA or any other type of polynucleotide, provided that the sequence can form a functional silencing complex to degrade the target mRNA transcript.

In one embodiment, the sequence comprises or consists of a short interfering RNA (siRNA). The siRNA can be, for example, dsRNA having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence-specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric dinucleotide 3′ overhangs. Using synthetic 21 bp siRNA duplexes (e.g., 19 RNA bases followed by a UU or dTdT 3′ overhang), sequence-specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression, since they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long double-stranded RNAs (dsRNAs) are first expressed from a tissue-specific promoter in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific promoters to create tissue-specific knockdown mice.

Any 3′ dinucleotide overhang, such as UU, can be used for siRNA design. In some cases, G residues in the overhang are avoided because of the potential for the siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of>4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA is designed to have two inverted repeats separated by a short spacer sequence and end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see, e.g., Sui et al, Proc. Natl. Acad. Sci. USA 99: 5515-5520, 2002), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide /RNase H method (Lee et al, Nature Biotechnology 20:500-505, 2002).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one example, the 5′ overhang includes about 6 nucleotide residues.

In still yet another embodiment, the target sequences for RNAi are about 21-mer sequence fragments selected from LRG coding sequences, such as SEQ ID NOS:1-29. The target sequences can be selected from either ORF regions or non-ORF regions. The 5′ end of each target sequence has dinucleotide “NA,” where “N” can be any base and “A” represents adenine. The remaining 19-mer sequence has a GC content of between 30% and 65%. In many examples, the remaining 19-mer sequence does not include any four consecutive A or T (i.e., AAAA or TTTT), three consecutive G or C (i.e., GGG or CCC), or seven “GC” in a row. Examples of the target sequences prepared using the above-described criteria (“Relaxed Criteria”) are illustrated in Table 3. Each target sequence in Table 3 has SEQ ID NO:3n+1, and the corresponding siRNA sense and antisense strands have SEQ ID NO:3n+2 and SEQ ID NO:3n+3, respectively, where n is a positive integer. For each LRG coding sequence (e.g., SEQ ID NOS:1-29), multiple target sequences can be selected.

Additional criteria can be used for RNAi target sequence design. In one example, the GC content of the remaining 19-mer sequence is limited to between 35% and 55%, and any 19-mer sequence having three consecutive A or T (i.e., AAA or TTT) or a palindrome sequence with 5 or more bases is excluded. In addition, the 19-mer sequence can be selected to have low sequence homology to other human genes. In one embodiment, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer sequences producing no hit to other human genes under the BLASTN search can be selected. During the search, the e-value may be set at a stringent value (such as “1”). Furthermore, the target sequence can be selected from the ORF region, and is at least 75-bp frorii the start and stop codons. Examples of the target sequences prepared using these criteria (“Stringent Criteria”) are demonstrated in Table 3 (SEQ ID NO:3n+1, where n is a positive integer). siRNA sense and antisense sequences (SEQ ID NO:3n+2 and SEQ ID NO:3n+3, respectively) for each target sequence (SEQ ID NO:3n+1) are also provided.

TABLE 3
RNAi Target Sequences and siRNA Sequences

| | Relaxed Criteria | Stringent Criteria |
|---|---|---|
| | (target: SEQ ID NO: 3n + 1; | (target: SEQ ID NO: 3n + 1; |
| SEQ ID NO | siRNA sense: SEQ ID NO: 3n + 2; | siRNA sense: SEQ ID NO: 3n + 2; |
| (LRG coding seq.) | siRNA antisense: SEQ ID NO: 3n + 3) | siRNA antisense: SEQ ID NO: 3n + 3) |
| SEQ ID NO: 1 | SEQ ID NOS: 58-390 | SEQ ID NOS: 391-414 |
| SEQ ID NO: 2 | SEQ ID NOS: 415-2,277 | SEQ ID NOS: −2,278-2,670 |
| SEQ ID NO: 3 | SEQ ID NOS: 2,671-2,778 | |
| SEQ ID NO: 4 | SEQ ID NOS: 2,779-2,961 | SEQ ID NOS: 2,962-2,982 |
| SEQ ID NO: 5 | SEQ ID NOS: 2,983-3,246 | SEQ ID NOS: 3,247-3,291 |
| SEQ ID NO: 6 | SEQ ID NOS: 3,292-3,948 | SEQ ID NOS: 3,949-3,993 |
| SEQ ID NO: 7 | SEQ ID NOS: 3,994-4,677 | SEQ ID NOS: 4,678-4,731 |
| SEQ ID NO: 8 | SEQ ID NOS: 4,732-5,388 | SEQ ID NOS: 5,389-5,439 |
| SEQ ID NO: 9 | SEQ ID NOS: 5,440-6,345 | SEQ ID NOS: 6,346-6,372 |
| SEQ ID NO: 10 | SEQ ID NOS: 6,373-8,514 | SEQ ID NOS: 8,515-8,664 |
| SEQ ID NO: 11 | SEQ ID NOS: 8,665-10,740 | SEQ ID NOS: 10,741-10,884 |
| SEQ ID NO: 12 | SEQ ID NOS: 10,885-11,151 | SEQ ID NOS: 11,152-11,157 |
| SEQ ID NO: 13 | SEQ ID NOS: 11,158-11,346 | SEQ ID NOS: 11,347-11,349 |
| SEQ ID NO: 14 | SEQ ID NOS: 11,350-11,691 | SEQ ID NOS: 11,692-11,703 |
| SEQ ID NO: 15 | SEQ ID NOS: 11,704-12,795 | SEQ ID NOS: 12,796-12,810 |
| SEQ ID NO: 16 | SEQ ID NOS: 12,811-13,014 | SEQ ID NOS: 13,015-13,032 |
| SEQ ID NO: 17 | SEQ ID NOS: 13,033-13,287 | SEQ ID NOS: 13,288-13,308 |
| SEQ ID NO: 18 | SEQ ID NOS: 13,309-13,512 | SEQ ID NOS: 13,513-13,533 |
| SEQ ID NO: 19 | SEQ ID NOS: 13,534-14,484 | SEQ ID NOS: 14,485-14,508 |
| SEQ ID NO: 20 | SEQ ID NOS: 14,509-15,240 | SEQ ID NOS: 15,241-15,258 |
| SEQ ID NO: 21 | SEQ ID NOS: 15,259-15,624 | |
| SEQ ID NO: 22 | SEQ ID NOS: 15,625-15,954 | |
| SEQ ID NO: 23 | SEQ ID NOS: 15,955-16,299 | |
| SEQ ID NO: 24 | SEQ ID NOS: 16,300-16,653 | |
| SEQ ID NO: 25 | SEQ ID NOS: 16,654-17,712 | SEQ ID NOS: 17,713-17,832 |
| SEQ ID NO: 26 | SEQ ID NOS: 17,833-18,900 | SEQ ID NOS: 18,901-19,008 |
| SEQ ID NO: 27 | SEQ ID NOS: 19,009-19,806 | SEQ ID NOS: 19,807-19,842 |
| SEQ ID NO: 28 | SEQ ID NOS: 19,843-20,673 | SEQ ID NOS: 20,674-20,751 |
| SEQ ID NO: 29 | SEQ ID NOS: 20,752-21,102 | SEQ ID NOS: 21,103-21,135 |

The effectiveness of the siRNA sequences can be evaluated using various methods known in the art. For instance, an siRNA sequence of the present invention can be introduced into a cell that over-expresses an LRG. The polypeptide or mRNA level of the LRG in the cell can be detected. A substantial change in the expression level of the LRG before and after the introduction of the siRNA sequence is indicative of the effectiveness of the siRNA sequence in suppressing the expression of the LRG. In one example, the expression levels of other genes are also monitored before and after the introduction of the siRNA sequence. An siRNA sequence which has inhibitory effect on the LRG expression but does not significantly affect the expression of other genes can be selected. In another example, multiple siRNA or other RNAi sequences can be introduced into the same target cell. These siRNA or RNAi sequences specifically inhibit the LRG gene expression but not the expression of other genes. In yet another example, siRNA or other RNAi sequences that inhibit the expression of both the LRG gene and other gene or genes can be used.

In yet another embodiment, the polynucleotide molecules of the present invention can be modified at the base moiety, sugar moiety or phosphate backbone to improve, e.g., the stability, hybridization, or solubility of the molecule. For example, the deoxyribose phosphate backbone of the polynucleotide molecules can be modified to generate peptide polynucleotides. As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols.

PNAs can be used in therapeutic and diagnostic applications. For example, PNAs can be used as antisense agents for sequence-specific modulation of LRG expression by, for example, inducing transcription or translation arrest or inhibiting replication. PNAs of the polynucleotide molecules of the invention can also be used in the analysis of single base pair mutations in a gene e.g., by PNA-directed PCR clamping, as artificial restriction enzymes when used in combination with other enzymes (e.g., S1 nucleases) or as probes or primers for DNA sequencing or hybridization.

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the poiynucleotide molecules of the invention can be generated which may combine the advantageous properties of PNA and DNA. Such chimeras allow DNA recognition enzymes, (e.g., DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation. The synthesis of PNA-DNA chimeras can be performed. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a spacer between the PNA and the 5′ end of DNA. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

In other embodiments, the oligonucleotide may include other appended groups such as peptides (e.g., for targeting host cell receptors in vivo), or agents facilitating transport across the cell membrane or the blood-kidney barrier (see, e.g., PCT Publication No. W089/10134). In addition, oligonucleotides can be modified with hybridization-triggered cleavage agents or intercalating agents. To this end, the oligonucleotide may be conjugated to another molecule (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label).

### Isolated Polypeptides

Several aspects of the invention pertain to isolated LRPPs and mutated LRPPs capable of inhibiting normal LRPP activity, as well as polypeptide fragments suitable for use as immunogens to raise anti-LRPP antibodies. In one embodiment, native LRPPs can be isolated from cells or tissue sources by an appropriate purification scheme using standard protein purification techniques. The degree of purification necessary will vary depending on the use of the LRPP. In some instances, no purification will be necessary.

In another embodiment, LRPPs or mutated LRPPs capable of inhibiting normal LRPP activity (dominant-negative mutants) are produced by recombinant DNA techniques. Alternatively, an LRPP or mutated LRPP can be synthesized chemically using standard peptide synthesis techniques.

The invention provides polypeptides encoded by human LRGs, such as SEQ ID NOS: 30-57. The invention also provides polypeptides that are substantially homologous to an LRPP, retaining the functional activity of the LRPP yet differing in amino acid sequence due to natural allelic variation or mutagenesis. In one embodiment, the LRPPs are variants which have at least about 60%, 65%, 70%, 75%, 80%, 85%, 90%, 95%, 98% or more sequence identity or similarity to the original LRPPs (e.g., SEQ ID NOS: 30-57) or the fragments thereof.

To determine the percent identity or similarity of two amino acid sequences or two nucleotide sequences, the sequences are aligned for optimal comparision purposes (e.g., gaps can be introduced in one or both of a first and second amino acid or polynucleotide sequence for optimal alignment and non-homologous sequences can be disregarded for comparison purposes). The percent identity or similarity between two sequences is a function of the number of identical or similar positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol., 48:444-453, 1970) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6.

The polynucleotide and protein sequences of the present invention can further be used as a “query sequence” to perform a search against public databases to, for example, identify other family members or related sequences. Such searches can be performed using the BLAST programs available at the BLAST website maintained by the National Center for Biotechnology Information at the National Institute of Health, Washington, DC.

The invention also provides chimeric or fusion LRPP. A fusion LRPP contains an LRG-related polypeptide and a non-LRG polypeptide fused in-frame to each other. The LRG-related polypeptide corresponds to all or a portion of an LRPP or its variant. In one embodiment, a fusion LRPP comprises at least one portion of an LRPP sequence recited in one of SEQ ID NOS:14-26.

A peptide linker sequence may be employed to separate the LRG-related polypeptide from non-LRG polypeptide components by a distance sufficient to ensure that each polypeptide folds into its secondary and tertiary structures. Such a peptide linker sequence is incorporated into the fusion protein using standard techniques well-known in the art. Suitable peptide linker sequences may be chosen based on the following factors (1) their ability to adopt a flexible extended conformation; (2) their inability to adopt a secondary structure that could interact with functional epitopes on the LRG-related polypeptide and non-LRG polypeptide, and (3) the lack of hydrophobic or charged residues that might react with the polypeptide functional epitopes. Exemplary peptide linker sequences contain gly, asn and ser residues. Other near neutral amino acids, such as thr and ala may also be used in the linker sequence. Amino acid sequences which may be usefully employed as linkers include those disclosed in Maratea et al., Gene, 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the LRG-related polypeptide and non-LRG polypeptide have non-essential N-terminal amino acid regions that can be used to separate the functional domains and prevent steric interference.

For example, in one embodiment, the fusion protein is a glutathione s-transferase (GST)-LRPP fusion protein in which the LRG-related sequences are fused to the C-terminus of the GST sequences. Such fusion proteins can facilitate the purification of recombinant LRPPs.

The LRPP-fusion proteins of the invention can be incorporated into pharmaceutical compositions and administered to a subject in vivo, as described herein. The LRPP-fusion proteins can be used to affect the bioavailability of an LRPP substrate. The LRPP-fusion proteins may be useful therapeutically for the treatment of or prevention of damage caused by, for example, (i) aberrant modification or mutation of an LRPP, and (ii) aberrant post-translational modification of an LRPP. It is also conceivable that a fusion protein containing a normal or mutated LRPP, or a fragment thereof, may be capable of inhibiting normal LRPP activity in a subject.

Moreover, the LRPP-fusion proteins can be used as immunogens to produce anti-LRPP antibodies in a subject, to purify LRPP ligands and in screening assays to identify molecules which inhibit the interaction of an LRPP with an LRPP substrate.

LRPP-fusion proteins used as immunogens may comprise a non-LRPP immunogenic protein. In one embodiment, the immunogenic protein is capable of eliciting a recall response.

In another embodiment, an LRPP-fusion protein of the invention is produced by standard recombinant DNA techniques. For example, DNA fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). An LRG-related polynucleotide can be cloned into such an expression vector such that the fusion moiety is linked in-frame to the LRG-related polypeptide.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence which facilitates purification, such as with a GST domain.

The present invention also pertains to variants of an LRPP which function as antagonists to the LRPP. In one embodiment, antagonists or agonists of LRPPs are used as therapeutic agents. For example, antagonists to an LRPP can decrease the activity of the LRPP and ameliorate SLE/LN in a subject wherein said LRPP is over-expressed. Variants of LRPPs can be generated by mutagenesis, e.g., discrete point mutation or truncation of an LRG.

In certain embodiments, an antagonist of an LRPP can inhibit one or more of the activities of the naturally occurring form of the LRPP by, for example, competitively modulating an activity of the LRPP. Thus, specific biological effects can be elicited by treatment with a variant of limited function.

Mutants of an LRPP which function as either LRPP agonists or as LRPP antagonists can be identified by screening combinatorial libraries of mutants. In certain embodiments, such variants may be used, for example, as a therapeutic protein of the invention. A variegated library of LRPP variants can be produced by, for example, enzymatically ligating a mixture of synthetic oligonucleotides into gene sequences such that a degenerate set of potential LRPP sequences is expressible as individual polypeptides, or alternatively, as a set of larger fusion proteins (e.g., for phage display) containing the set of LRPP sequences therein. There are a variety of methods which can be used to produce libraries of potential LRPP variants from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential LRPP sequences. Methods for synthesizing degenerate oligonucleotides are known in the art.

In addition, libraries of fragments of a protein coding sequence corresponding to an LRG can be used to generate a variegated population of LRPP fragments for screening and subsequent selection of variants of an LRPP. In one embodiment, a library of coding sequence fragments can be generated by treating a double-stranded PCR fragment of an LRG coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, an expression library can be derived which encodes N-terminal, C-terminal and internal fragments of various sizes of the LRPP.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify LRPP variants (Delgrave et al., Protein Engineering, 6:327-331, 1993).

Portions of an LRPP or variants of an LRPP having less than about 100 amino acids, and generally less than about 50 amino acids, may also be generated by synthetic means, using techniques well-known to those of ordinary skill in the art. For example, such polypeptides may be synthesized using any of the commercially available solid-phase techniques, such as the Merrifield solid-phase synthesis method, where amino acids are sequentially added to a growing amino acid chain. Equipment for automated synthesis of polypeptides is commercially available from suppliers such as Perkin Elmer/Applied BioSystems Division (Foster City, Calif.), and may be operated according to the manufacturer's instructions.

Methods and compositions for screening for protein inhibitors or activators are known in the art (see U.S. Patent Nos. 4,980,281, 5,266,464, 5,688,635, and 5,877,007, which are incorporated herein by reference).

### Antibodies

In accordance with another aspect of the present invention, antibodies specific to an LRPP or its variants are prepared. In many embodiments, an antibody of the present invention can bind to an LRPP with a binding affinity of at least 10⁵, 10⁶, 10⁷ M⁻¹ or more. The antibodies can be monoclonal, polyclonal, chimeric, or humanized antibodies.

In another aspect, the invention provides methods of making an isolated hybridoma which produces an antibody useful for diagnosing a patient or animal with SLE/LN. In this method, an LRPP or its variant is isolated (e.g., by purification from a cell in which it is expressed or by transcription and translation of a polynucleotide encoding the protein in vivo or in vitro using known methods). A vertebrate, such as a mammal (e.g., a mouse, rabbit or sheep), can be immunized using the isolated polypeptide or polypeptide fragment. The vertebrate may optionally be immunized at least one additional time with the isolated polypeptide or polypeptide fragment, so that the vertebrate exhibits a robust immune response to the polypeptide or polypeptide fragment. Splenocytes are isolated from the immunized vertebrate and fused with an immortalized cell line to form hybridomas, using any of a variety of methods well-known in the art. Hybridomas formed in this manner are then screened using standard methods to identify one or more hybridomas which produce an antibody which specifically binds with the polypeptide or polypeptide fragment. The invention also includes hybridomas made by this method and antibodies made using such hybridomas.

An isolated LRPP, or a portion or fragment thereof, can be used as an immunogen to generate antibodies that bind the LRPP using standard techniques for polyclonal and monoclonal antibody preparation. A full-length LRPP can be used or, alternatively, the invention provides antigenic peptide fragments of the LRPP for use as immunogens. The antigenic peptide of an LRPP comprises at least 8 amino acid residues of an amino acid sequence encoded by an LRG, and encompasses an epitope of an LRPP such that an antibody raised against the peptide forms a specific immune complex with the LRPP. The antigenic peptide can include, without limitation, at least 8, 12, 16, 20 or more amino acid residues.

Immunogenic portions (epitopes) may generally be identified using well-known techniques. Such techniques include screening polypeptides for the ability to react with antigen-specific antibodies, antisera and/or T-cell lines or clones. Such antisera and antibodies may be prepared as described herein, and using well-known techniques. An epitope of an LRPP is a portion that reacts with such antisera and/or T-cells at a level that is not substantially less than the reactivity of the full length polypeptide (e.g., in an ELISA and/or T-cell reactivity assay). Such epitopes may react within such assays at a level that is similar to or greater than the reactivity of the full length polypeptide. Such screens may generally be performed using methods well-known to those of ordinary skill in the art. For example, a polypeptide may be immobilized on a solid support and contacted with patient sera to allow the binding of antibodies within the sera to the immobilized polypeptide. Unbound sera may then be removed and bound antibodies detected using, for example, ¹²⁵I-labeled Protein A.

Exemplary epitopes encompassed by the antigenic peptide are regions of an LRPP that are located on the surface of the polypeptide, e.g., hydrophilic regions, as well as regions with high antigenicity.

An LRPP immunogen typically is used to prepare antibodies by immunizing a suitable subject, (e.g., rabbit, goat, mouse or other mammal) with the immunogen. An appropriate immunogenic preparation can contain, for example, recombinantly expressed LRPP or a chemically synthesized LRPP. The preparation can further include an adjuvant, such as Freund's complete or incomplete adjuvant, or similar immunostimulatory agent. Immunization of a suitable subject with an immunogenic LRPP preparation induces a polyclonal anti-LRPP antibody response. Techniques for preparing, isolating and using antibodies are well-known in the art.

Accordingly, another aspect of the invention pertains to monoclonal or polyclonal anti-LRPP antibodies. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to an LRPP.

Polyclonal anti-LRPP antibodies can be prepared as described above by immunizing a suitable subject with an LRPP. The anti-LRPP antibody titer in the immunized subject can be monitored over time by standard techniques, such as with an enzyme linked immunosorbent assay (ELISA) using immobilized LRPP or a fragment of LRPP. If desired, the antibody molecules directed against LRPP can be isolated from the mammal (e.g., from the blood) and further purified by well-known techniques, such as protein A chromatography, to obtain the lgG fraction. At an appropriate time after immunization, e.g., when the anti-LRPP antibody titers are the highest, antibody-producing cells can be obtained from the subject and used to prepare monoclonal antibodies by standard techniques, such as the hybridoma technique, human B cell hybridoma technique, the EBV-hybridoma technique, or trioma techniques. The technology for producing monoclonal antibody hybridomas is well-known. Briefly, an immortal cell line (typically a myeloma) is fused to lymphocytes (typically splenocytes) from a mammal immunized with an LRPP immunogen as described above, and the culture supernatants of the resulting hybridoma cells are screened to identify a hybridoma producing a monoclonal antibody that binds to an LRPP.

Any of the many well-known protocols used for fusing lymphocytes and immortalized cell lines can be applied for the purpose of generating an anti-LRPP monoclonal antibody. Moreover, the ordinarily skilled worker will appreciate that there are many variations of such methods which also would be useful.

Alternative to preparing monoclonal antibody-secreting hybridomas, a monoclonal anti-LRPP antibody can be identified and isolated by screening a recombinant combinatorial immunoglobulin library (e.g., an antibody phase display library) with an LRPP to thereby isolate immunoglobulin library members that bind to the LRPP. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612).

The anti-LRPP antibodies also include “single-chain Fv” or “scFv” antibody fragments. The scFv fragments comprise the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding.

Additionally, recombinant anti-LRPP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In one embodiment, humanized antibodies are used for therapeutic treatment of human subjects. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies), which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. The humanized antibody may also comprise at least a portion of an immunoglobulin constant region (Fc), typically that of a human immunoglobulin.

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of an LRPP. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope.

In an embodiment, the antibodies to an LRPP are capable of reducing or eliminating the biological function of the LRPP. In one example, at least a 25% decrease in activity is achieved. In another embodiment, at least about 50%, 60%, 70%, 80%, 90%, or more decrease in activity is obtained.

An anti-LRPP antibody can be used to isolate the LRPP by standard techniques, such as affinity chromatography or immunoprecipitation. An anti-LRPP antibody can facilitate the purification of a natural LRPP from cells and of a recombinantly produced LRPP expressed in host cells. Moreover, an anti-LRPP antibody can be used to detect an LRPP (e.g., in a cellular lysate or cell supernatant on the cell surface) in order to evaluate the abundance and pattern of expression of the LRPP. Anti-LRPP antibodies can be used diagnostically to monitor protein levels in tissue as part of a clinical testing procedure to, for example, determine the efficacy of a given treatment regimen. Detection can be facilitated by directly or indirectly coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S and 3H.

Anti-LRPP antibodies of the invention are also useful for targeting a therapeutic to a cell or tissue comprising the antigen of the anti-LRPP antibody. For example, a therapeutic such as a small molecule can be linked to the anti-LRPP antibody in order to target the therapeutic to the cell or tissue comprising the LRPP antigen.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfbydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter el al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler el al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used.

### Vectors

Another aspect of the invention pertains to vectors containing polynucleotides encoding LRPPs or portions thereof. Vectors can be plasmids or viral vectors.

The expression vectors of the invention can be designed for expression of LRPPs in prokaryotic or eukaryotic cells. For example, LRPPs can be expressed in bacterial cells such as E. coli, insect cells (using baculovirus expression vectors), yeast cells, or mammalian cells. In certain embodiments, such protein may be used, for example, as a therapeutic protein of the invention. Alternatively, the expression vector can be transcribed and translated in vitro, for example using T7 promoter regulatory sequences and T7 polymerase.

In another embodiment, mammalian expression vector including tissue-specific regulatory elements are used to express the polynucleotides of interest. Tissue-specific regulatory elements are known in the art and may include epithelial cell-specific promoters. Other non-limiting examples of suitable tissue-specific promoters include the liver-specific albumin promoter, lymphoid-specific promoters, promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also encompassed, for example the α-fetoprotein promoter.

The LRGs identified in the present invention can be used for therapeutic purposes. For example, antisense constructs of the LRGs can be delivered therapeutically to SLE/LN cells. The goal of such therapy is to retard the growth rate of the SLE/LN-affected cells. Expression of the sense molecules and their translation products or expression of the antisense mRNA molecules has the effect of inhibiting the growth rate of SLE/LN-affected cells.

The invention also provides a recombinant expression vector comprising a polynucleotide encoding a LRPP cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to an LRG of the invention. Regulatory sequences operatively linked to a polynucleotide cloned in the antisense orientation can be chosen to direct the continuous expression of the antisense RNA molecule in a variety of cell types. For instance viral promoters or enhancers, or regulatory sequences can be chosen to direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region. The activity of the promoter/enhancer can be determined by the cell type into which the vector is introduced.

The invention further provides gene delivery vehicles for delivery of polynucleotides to cells, tissues, or a mammal for expression. For example, a polynucleotide sequence of the invention can be administered either locally or systemically in a gene delivery vehicle. These constructs can utilize viral or non-viral vector approaches in in vivo or ex vivo modality. Expression of such coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle can be, for example, a viral vector, such as a retroviral, lentiviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector.

Delivery of the gene therapy constructs of this invention into cells is not limited to the above mentioned viral vectors. Other delivery methods and media may be employed such as, for example, nucleic acid expression vectors, polycationic condensed DNA linked or unlinked to killed adenovirus alone, ligand linked DNA, liposome-DNA complex, eukaryotic cell delivery vehicles cells, deposition of photopolymerized hydrogel materials, handheld gene transfer particle gun, ionizing radiation, nucleic charge neutralization or fusion with cell membranes. Particle mediated gene transfer may be employed. Briefly, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose or transferrin. Naked DNA may also be employed. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Another aspect of the invention pertains to the expression of LRGs using a regulatable expression system. These systems include, but are not limited to, the Tet-on/off system, the Ecdysone system, the Progesterone-system, and the Rapamycin-system.

Another aspect of the invention pertains to the use of host cells which are transformed, transfected, or transduced with vectors encoding or comprising LRGs or portions thereof. The host cells can be prokaryotic or eukaryotic cells. These host cells can be employed to express any desired LRPP.

### Detection Methods

As discussed earlier, expression level of LRG may be used as a marker for SLE/LN. Detection and measurement of the relative amount of an LRG product (polynucleotides or polypeptides) can be by any method known in the art.

Typical methodologies for detection of a transcribed polynucleotide include extraction of RNA from a cell or tissue sample, followed by hybridization of a labeled probe to the extracted RNA and detection of the labeled probe (e.g., Northern blotting, or nucleic acid array).

Typical methodologies for peptide detection include protein extraction from a cell or tissue sample, followed by binding of an antibody specific for the target protein to the protein sample, and detection of the antibody. For example, detection of an LRPP may be accomplished using an anti-LRPP polyclonal antibody. Antibodies are generally detected by the use of a labeled secondary antibody. The label can be a radioisotope, a fluorescent compound, an enzyme, an enzyme co-factor, or ligand. Such methods are well understood in the art.

In certain embodiments, the LRG itself may serve as a marker for SLE/LN. For example, an increase or decrease of genomic copies of an LRG, such as by duplication or deletion of the gene, may be correlated with SLE/LN.

Detection of specific polynucleotide molecules may also be assessed by gel electrophoresis, column chromatography, or direct sequencing, quantitative PCR, RT-PCR, nested-PCR, or other techniques known in the art.

Detection of the presence or number of copies of all or a part of an LRG may be performed using any method known in the art. In one embodiment, Southern analysis is employed to assess the presence and/or quantity of the genomic copies of the LRG. Other useful methods for DNA detection and/or quantification include, but are not limited to, direct sequencing, gel electrophoresis, column chromatography, quantitative PCR, or other means as appreciated by those skilled in the art.

### Screening Methods

The invention also provides methods (such as screening assays) for identifying modulators, such as candidate or test compounds or agents comprising therapeutic moieties (e.g., peptides, peptidomimetics, peptoids, polynucleotides, small molecules or drugs) which can (a) bind to an LRPP, (b) have a modulatory (e.g., stimulatory or inhibitory) effect on the activity of an LRPP, .(c) have a modulatory effect on the interactions of an LRPP with one or more of its natural substrates (e.g., peptides, proteins, hormones, co-factors, or polynucleotides), or (d) have a modulatory effect on the expression of an LRG. Such assays typically comprise a reaction between an LRPP and one or more assay components. The other components may be either the test compound itself, or a combination of test compound and a binding partner of an LRPP.

The test compounds of the present invention are generally inorganic molecules, small organic molecules, and biomolecules. Biomolecules include, but are not limited to, polypeptides, polynucleotides, polysaccharides, as well as any naturally-occurring or synthetic organic compounds that have a bioactivity in mammals. In one embodiment the test compound is a small organic molecule. In another embodiment, the test compound is a biomolecule.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., J. Med. Chem., 37: 2678-85, 1994); spatially addressable parallel solid phase or solution phase libraries; synthetic library methods requiring deconvolution the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des., 12:145, 1997).

As used herein, the term “binding partner” refers to a bioactive agent which serves as either a substrate for an LRPP, or alternatively, as a ligand having binding affinity to an LRPP. As mentioned above, the bioactive agent may be any of a variety of naturally-occurring or synthetic compounds proteins, peptides, polysaccharides, nucleotides or polynucleotides.

### Screening for Inhibitors of LRPPs

The invention provides methods of screening test compounds for inhibitors of an LRPP, and of screening for the pharmaceutical compositions comprising the test compounds. The method of screening comprises obtaining samples from subjects diagnosed with or suspected of having SLE/LN, contacting each separate aliquot of the samples with one of a plurality of test compounds, and comparing the expression of LRGs in each of the aliquots to determine whether any of the test compounds provides a substantially decreased level of expression or activity of LRGs relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating the binding of an LRPP to a binding partner, by combining the test compound, LRPP, and binding partner together and determining whether binding of the binding partner and the LRPP occurs. The test compound may be either small molecules or a bioactive agent. As discussed below, test compounds may be provided from a variety of libraries well-known in the art.

Inhibitors of LRG expression, activity or binding ability are useful as therapeutic compositions of the invention. Such inhibitors may be formulated as pharmaceutical compositions, as described herein below. Such modulators may also be used in the methods of the invention, for example, to diagnose, treat, or prognose SLE/LN.

### High-Throughout Screening Assays

The invention provides methods of conducting high-throughput screening for test compounds capable of inhibiting the activity or expression of LRGs. In one embodiment, the method of high-throughput screening involves combining test compounds and an LRPP and detecting the effect of the test compound on the LRPP. Functional assays such as cytosensor microphysiometer, calcium flux assays such as FLIPR® (Molecular Devices Corp, Sunnyvale, Calif.), or the TUNEL assay may be employed to measure cellular activity, as discussed below.

A variety of high-throughput functional assays well-known in the art may be used in combination to screen and/or study the reactivity of different types of activating test compounds. Since the coupling system is often difficult to predict, a number of assays may need to be configured to detect a wide range of coupling mechanisms. A variety of fluorescence-based techniques are well-known in the art and are capable of high-throughput and ultra-high throughput screening for activity, including but not limited to BRET® or FRET® (both by Packard instrument Co., Meriden, Conn.). The ability to screen a large volume and a variety of test compounds with great sensitivity permits for analysis of the therapeutic targets of the invention to further provide potential inhibitors of SLE/LN. The BIACORE® system may also be manipulated to detect binding of test compounds with individual components of the therapeutic target.

By combining test compounds with an LRPP and determining the binding activity between them, diagnostic analysis can be performed to elucidate the coupling systems. Generic assays using a cytosensor microphysiometer may also be used to measure metabolic activation, while changes in calcium mobilization can be detected by using the fluorescence—based techniques such as FLPR® (Molecular Devices Corp, Sunnyvale, Calif.). In addition, the presence of apoptotic cells may be determined by the TUNEL assay, which utilizes flow cytometry to detect free 3-OH termini resulting from cleavage of genomic DNA during apoptosis. As mentioned above, a variety of functional assays well-known in the art may be used in combination to screen and/or study the reactivity of different types of activating test compounds. In one embodiment, the high-throughput screening assay of the present invention utilizes label-free plasmon resonance technology as provided by the BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit an activity of an LRPP, by combining the test compounds and the LRPP in high-throughput assays such as BIACORE®, or in fluorescence-based assays such as BRET®. In addition, high-throughput assays may be utilized to identify specific factors which bind to an LRPP, or alternatively, to identify test compounds which prevent binding of an LRPP to the binding partner. Moreover, the high-throughput screening assays may be modified to determine whether test compounds can bind to either an LRPP or to a binding partner of the LRPP.

### Detection of Genetic AIterations

The methods of the invention can also be used to detect genetic alterations in an LRG, thereby determining if a subject with the altered gene is at risk for damage characterized by aberrant regulation in LRG activity or polynucleotide expression. In one embodiments, the methods include detecting, in a sample of cells from the subject, the presence or absence of a genetic alteration characterized by at least one alteration affecting the integrity of an LRG, or the aberrant expression of the LRG. For example, such genetic alterations can be detected by ascertaining the existence of at least one of the following: (i) deletion of one on more nucleotides from an LRG; (ii) addition of one or more nucleotides to an LRG; (iii) substitution of one or more nucleotides of an LRG; (iv) a chromosomal rearrangement of an LRG; (v) alteration in the level of a messenger RNA transcript of an LRG; (vi) aberrant modification of an LRG, such as of the methylation pattern of the genomic DNA; (vii) the presence of a non-wild-type splicing pattern of a messenger RNA transcript of an LRG; (viii) non-wild-type level LRG; (ix) allelic loss of an LRG, and (x) inappropriate post-translational modification of LRG products. As described herein, there are a large number of assays known in the art, which can be used for detecting alterations in an LRG. An example of a biological sample is a blood sample isolated by conventional means from a subject.

In certain embodiments, detection of the alteration involves the use of a probe/primer in a polymerase chain reaction (PCR), such as anchor PCR or RACE PCR, or, alternatively, in a ligation chain reaction (LCR), the latter of which can be particularly useful for detecting point mutations in the LRG. This method can include the steps of collecting a sample of cells from a subject, isolating a polynucleotide (e.g., genomic, mRNA or both) from the cells of the sample, contacting the polynucleotide sample with one or more primers which specifically hybridize to an LRG under conditions such that hybridization and amplification of the LRG (if present) occurs, and detecting the presence or absence of an amplification product, or detecting the size of the amplification product and comparing the length to a control sample. It is understood that PCR and/or LCR may be desirable to use as a preliminary amplification step in conjunction with any of the techniques used for detecting mutations described herein.

Alternative amplification methods include: self-sustained sequence replication, transcriptional amplification system, Q-Beta Replicase, or any other polynucleotide amplification method, followed by the detection of the amplified molecules using techniques well-known to those of skill in the art. These detection schemes are especially useful for the detection of polynucleotide molecules if such molecules are present in very low numbers.

In an alternative embodiment, mutations in an LRG from a sample cell can be identified by alterations in restriction enzyme cleavage patterns. For example, samples and control DNA are isolated, amplified (optionally), digested with one or more restriction endonucleases, and fragment length sizes are determined by gel electrophoresis and compared. Differences in fragment length sizes between sample and control DNA indicate mutations in the sample DNA. Moreover, the use of sequence specific ribozymes can be used to score for the presence of specific mutations by development or loss of a ribozyme cleavage site.

In other embodiments, genetic mutations in an LRG can be identified by hybridizing sample and control polynucleotides, e.g., DNA or RNA, to high density arrays containing LRG cDNAs obtained from samples of cells. For example, the mutY enzyme of E. coli cleaves A at G/A mismatches and the thymidine DNA glycosylase from HeLa cells cleaves T at G/T mismatches. According to an exemplary embodiment, a probe based on an LRG sequence, e g, a wild-type LRG sequence, is hybridized to cDNA or other DNA product from a test cell(s) The duplex is treated with a DNA mismatch repair enzyme, and the cleavage products, if any, can be detected from electrophoresis protocols or the like. See, for example, U.S. Pat. No 5,459,039.

In other embodiments, alterations in electrophoretic mobility will be used to identify mutations in an LRG. For example, single strand conformation polymorphism (SSCP) may be used to detect differences in electrophoretic mobility between mutant and wild-type polynucleotides single-stranded DNA fragments of sample and control LRG polynucleotides will be denatured and allowed to renature. The secondary structure of single-stranded polynucleotides varies according to sequence. The resulting alteration in electrophoretic mobility enables the detection of even a single base change. The DNA fragments may be labeled or detected with labeled probes. The sensitivity of the assay may be enhanced by using RNA (rather than DNA) in which the secondary structure is more sensitive to a change in sequence. In one embodiment, the subject method utilizes heteroduplex analysis to separate double-stranded heteroduplex molecules on the basis of changes in electrophoretic mobility (Keen el al., Trends Genet., 75, 1991).

In yet another embodiment the movement of mutant or wild-type fragments in polyacrylamide gels containing a gradient of denaturant is assayed using denaturing gradient gel electrophoresis (DGGE). When DGGE is used as the method of analysis, DNA will be modified to insure that it does not completely denature, for example, by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, Biophys. Chem., 265 12753, 1987).

Examples of other techniques for detecting point mutations include, but are not limited to selective oligonucleotide hybridization, selective amplification, and selective primer extension. For example, oligonucleotide primers may be prepared in which the known mutation is placed centrally and then hybridized to target DNA under conditions which permit hybridization only if a perfect match is found (Saiki et al., Proc Natl. Acad. Sci. USA, 86:6230, hundreds or thousands of oligonucleotides probes. For example, genetic mutations in an LRG can be identified in two-dimensional arrays containing light generated DNA probes. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the LRG and detect mutations by comparing the sequence of the sample LRG with the corresponding wild-type (control) sequence. It is also contemplated that any of a variety of automated sequencing procedures can be utilized when performing the diagnostic assays, including sequencing by mass spectrometry.

Other methods for detecting mutations in an LRG include methods in which protection from cleavage agents is used to detect mismatched bases in RNA/RNA or RNA/DNA heteroduplexes (Myers et al., Science, 230: 1242, 1985). In general, the art technique of “mismatch cleavage” starts by providing heteroduplexes by hybridizing (labeled) RNA or DNA containing the wild-type LRG sequence with potentially mutant RNA or DNA obtained from a tissue sample. The double-stranded duplexes are treated with an agent which cleaves single.-stranded regions of the duplex which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. In one embodiment, the control DNA or RNA can be labeled for detection.

In still another embodiment, the mismatch cleavage reaction employs one or more proteins that recognize mismatched base pairs in double-stranded DNA (so-called “DNA mismatch repair” enzymes) in defined systems for detecting and mapping point mutations in 1989). Such allele-specific oligonucleotides are hybridized to PCR amplified target or a number of different mutations when the oligonucleotides are attached to the hybridizing membrane and hybridized with labeled target DNA

Alternatively, allele-specific amplification technology which depends on selective PCR amplification may be used in conjunction with the instant invention Oligonucleotides used as primers for specific amplification may carry the mutation of interest in the center of the molecule (so that amplification depends on differential hybridization) or at the extreme 3′ end of one primer where, under appropriate conditions, mismatch can prevent or reduce polymerase extension. In addition, it may be desirable to introduce a novel restriction site in the region of the mutation to create cleavage-based detection It is anticipated that in certain embodiments amplification may also be performed using Taq ligase for amplification. In such cases, ligation will occur only if there is a perfect match at the 3′ end of the 5′ sequence making it possible to detect the presence of a known mutation at a specific site by looking for the presence or absence of amplification.

### Diagnostic and Prognostic Assays

The expression profile of an LRG or a panel of LRGs in a biological sample can be used for the diagnosis of SLE/LN. An exemplary diagnosis method includes the steps of obtaining .a biological sample from a test subject, contacting the biological sample with an agent capable of detecting an LRG product (e.g., an LRPP or an LRPN), determining expression level of the LRG product, and comparing the LRG expression in the biological sample to a reference level of LRG expression.

In one embodiment, the expression levels of one or more LRG products in a sample is compared to the expression levels in a normal sample, and an increased LRG expression/activity in the test sample indicates SLE/LN. A normal sample is a biological sample taken from a subject who is disease-free, who has not suffered from SLE/LN, or who is substantially free of SLE/LN. In another embodiment, the biological sample contains protein molecules from the test subject. Alternatively, the biological sample can contain mRNA molecules from the test subject. A biological sample from a subject can include a tissue sample, urine sample or blood sample. A tissue sample can be isolated by conventional means, e.g., a biopsy sample (such as a kidney or liver sample) from lupus patients. In many cases, the biological sample is a blood sample.

In one embodiment, the present invention provides a method for monitoring the effectiveness of treatment of a subject with an agent (e.g., an agonist, antagonist, peptidomimetic, protein, peptide, polynucleotide, small molecule, or other drug candidate identified by the screening assays described herein). The method includes the steps of (i) obtaining a pre-administration sample from a subject prior to administration of the agent; (ii) detecting the level of expression of an LRG in the pre-administration sample; (iii) obtaining a post-administration sample from the subject; (iv) detecting the level of expression of the LRG in the post-administration sample; and (v) comparing the level of expression of the LRG in the pre-administration sample with that in the post-administration sample. In order to optimize the treatment, the amount or frequency of administration of the agent can be adjusted. In many examples, reduction or elimination of abnormality in the expression of the LRG is indicative of the effectiveness of the agent.

In another embodiment, the effectiveness of an agent determined by a screening assay, as described herein to decrease LRG expression, protein levels, or down-regulate LRG protein activity, can be monitored in clinical trials of subjects exhibiting increased LRG expression, protein levels, or up-regulated LRG protein activity. In such clinical trials, the expression or activity of LRG can be used as a “read-out”, indicative of the physiological response of the cells to the agent. Accordingly, this response state may be determined before treatment and at various points during treatment of the individual with the agent.

The methods described herein may be performed, for example, by utilizing prepackaged diagnostic kits comprising at least one probe polynucleotide or antibody reagent described herein, which may be conveniently used, e.g., in clinical settings to diagnose or screen subjects for SLE/LN. Furthermore, any cell type or tissue in which an LRG is expressed may be utilized in the prognostic or diagnostic assays described herein.

In one embodiment, the prognostic or diagnostic assay analyzes the expression levels of 2, 3, 4, 5, 6, 7, or 8 LRGs selected from Table 1.

An exemplary agent for detecting an LRPP is an antibody capable of binding to the LRPP, such as an antibody with a detectable label. Antibodies can be polyclonal or monoclonal. An intact antibody, or a fragment thereof (e.g., Fab or F(ab′)₂) can be used. The term “labeled,” with regard to the probe or antibody, is intended to encompass direct labeling as well as indirect labeling (e.g., by reactivity with another reagent that is directly labeled). Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject.

The detection method of the invention can be used to detect LRG products in a biological sample in vitro as well as in vivo. For example, in vitro techniques for detection of LRG mRNA include Northern hybridizations, in situ hybridizations, RT-PCR, Taqman analysis, and biochip technology as described herein. In vitro techniques for detection of LRG protein include enzyme linked immunosorbent assays (ELISAs), Western blots, immunoprecipitation, immunofluorescence and biochip technology. Furthermore, in vivo techniques for detection of LRG expression include introducing into a subject a labeled anti-LRPP antibody.

The diagnostic method described herein can also be utilized to identify subjects having or at risk of developing SLE/LN associated with aberrant LRG expression or activity.

Prognostic assays can be devised to determine whether a subject undergoing treatment for SLE/LN has a poor outlook for long term survival or disease progression. In one embodiment, prognosis can be determined shortly after diagnosis, e.g., within a few days. By establishing LRG expression profiles of different stages of SLE/LN, from onset to later stages, an expression pattern may emerge to correlate a particular expression profile to increased likelihood of a poor prognosis. The prognosis may then be used to devise a more aggressive treatment program and enhance the likelihood of long-term survival and well-being.

The diagnostic assays may be used to determine the progression or seventy of SLE/LN before and after treatment The diagnostic assays may also be used to monitor effects during clinical trials.

In another embodiment, the reference expression levels of LRGs, such as the expression levels derived from disease-free humans or known SLE/LN patients, are stored in a database and are readily retrievable.

In yet another embodiment, the comparison between expression profiles of various genes is performed electronically, such as using a computer system. The computer system comprises a processor coupled to a memory which stores data representing the expression profiles being compared. In one example, the memory is readable as well as rewritable. The expression data stored in the memory can be changed, retrieved or otherwise manipulated. The memory also stores one or more programs capable of causing the processor to compare the stored expression profiles For instance, the program may be able to execute a weighted voting algorithm. The processor can also be coupled to a polynucleotide array scanner and is capable of receiving signals from the scanner.

The gene expression analysis of this invention can be used to identify genes that are differentially expressed in samples isolated at different stages of the progression, development or treatment of SLE/LN Genes thus-identified are molecular markers for monitoring the progression, development or treatment of SLE/LN Genes thus-identified can also be used as surrogate markers for evaluating the efficacy of a treatment for SLE/LN.

A clinical challenge concerning SLE/LN is the highly variable response of patients to therapy. The basic concept of pharmacogenomics is to understand a patient's genotype in relation to available treatment options and then individualize the most appropriate option for the patient. Different classes of SLE/LN patients can be created based on their different responses to a given therapy. Differentially expressed genes in these classes can be identified using the global gene expression analysis. Genes thus-identified can serve as predictive markers for forecasting whether a particular patient will be more or less responsive to the given therapy. For patients predicted to have a favorable outcome for the therapy, efforts to minimized toxicity of the therapy may be considered, whereas for those predicted not to respond to the therapy, treatment with other therapies or experimental regimes.

### Methods of Treatment

The present invention provides for both prophylactic and therapeutic methods of treating a subject at risk for, susceptible to or diagnosed with SLE/LN. With regard to both prophylactic and therapeutic methods of treatment, such treatments may be specifically tailored or modified, based on knowledge obtained from the field of pharmacogenomics. “Pharmacogenomics,” as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype” or “drug response genotype”). Thus, another aspect of the invention provides methods for tailoring an individual's prophylactic or therapeutic treatment with LRG modulators according to that individual's drug response. Pharmacogenomics allows a clinician or physician to target prophylactic or therapeutic treatments to subjects who will most benefit from the treatment and to avoid treatment of subjects who will experience toxic drug-related side effects.

### Prophylactic Methods

In one aspect, the invention provides a method for preventing in a subject SLE/LN associated with aberrant LRG expression or activity, by administering to the subject an agent which modulates LRG protein expression or activity.

Subjects at risk for SLE/LN which is caused or contributed to by aberrant LRG expression or activity can be identified by, for example, any or a combination of diagnostic or prognostic assays as described herein.

Administration of a prophylactic agent can occur prior to the manifestation of symptoms characteristic of the differential LRG protein expression, such that SLE/LN is prevented or, alternatively, delayed in its progression. Depending on the type of LRG aberrancy (e.g., typically a modulation outside the normal standard deviation), an LRG mutant protein, LRG protein antagonist agent, or LRG antisense polynucleotide, for example, can be used for treating the subject. The appropriate agent can be determined based on screening assays described herein.

### Therapeutic Methods

Another aspect of the invention pertains to methods of modulating LRG protein expression or activity for therapeutic purposes. Accordingly, in an exemplary embodiment, the modulatory method of the invention involves contacting a cell with an agent that inhibits LRG expression or one or more of the activities of an LRG protein associated with the cell. An agent that modulates LRG expression or protein activity can be an agent as described herein, such as a polynucleotide, a polypeptide, or a polysaccharide, a naturally-occurring target molecule of an LRG protein (e.g., an LRG protein substrate or receptor), an anti-LRPP antibody, an LRPP antagonist, a peptidomimetic of an LRG protein antagonist, or other small organic and inorganic molecule.

These modulatory methods can be performed in vivo (e.g., by administering the agent to a subject). As such, the present invention provides methods of treating an individual diagnosed with or at risk for SLE/LN characterized by aberrant expression or activity of an LRG. In. one embodiment, the method involves administering an agent (e.g., an agent identified by a screening assay described herein) or combination of agents that down-regulates LRG expression or activity. The agent may include a vector comprising a polynucleotide encoding an LRG inhibitor or an antisense sequence The agent may be an anti-LRPP antibody, a plurality of anti-LRPP antibodies or an anti-LRPP antibody conjugated to a therapeutic moiety. Treatment with the antibody may further be localized to the tissues or cells affected by SLE/LN.

### Pharmacogenomics

In conjunction with treatment for SLE/LN using an LRG modulator, pharmacogenomics analyses may be performed. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an LRG modulator as well as tailoring the dosage and/or therapeutic regimen of treatment with an LRG modulator

Pharmacogenomics deals with clinically significant hereditary variations in the response to drugs due to altered drug disposition and abnormal action in affected persons In general, two types of pharmacogenetic conditions can be differentiated Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is hemolysis after ingestion of oxidant drugs (anti-malarials, sulfonamides, analgesics, nitrofurans) and consumption of fava beans.

One pharmacogenomics approach to identifying genes that predict drug response, known as “a genome-wide association,” relies primarily on a high-resolution map of the human genome consisting of already known gene-related sites (e.g., a “bi-allelic” gene marker map which consists of 60,000-100,000 polymorphic or variable sites on the human genome, each of which has two variants). Such a high-resolution genetic map can be compared to a map of the genome of each of a statistically substantial number of subjects taking part in a Phase II/III drug trial to identify genes associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA A SNP may be involved in a disease process. However, the vast majority of SNPs may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals. Thus, mapping of the LRGs to SNP maps of LN patients may allow easier identification of these genes according to the genetic methods described herein.

Alternatively, a method termed the “candidate gene approach,” can be utilized to identify genes that predict drug response. According to this method, if a gene that encodes a drug target is known (e.g. an LRG), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer and poor metabolizer. The prevalence of poor metabolizer phenotypes is different among different populations. For example, the gene coding for CYP2D6 is highly polymorphic and several mutations have been identified in poor metabolizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabolizers show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., LRG expression in response to an LRG modulator ) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an LRG modulator.

### Pharmaceutical Compositions

The invention is further directed to pharmaceutical compositions comprising an LRG modulator and a pharmaceutically-acceptable carrier.

As used herein the language “pharmaceutically-acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it may be desirable to include isotonic agents, such as sodium chloride, sugars, polyalcohols (e.g., manitol, sorbitol, etc.) in the composition. Prolonged absorption of the injectable compositions can be brought about by including in the composition an agent which delays absorption, for example, aluminum monostearate and gelatin.

Sterile injectable solutions can be prepared by incorporating the active modulator (e.g., an anti-LRPP antibody, an LRG activity inhibitor, or a gene therapy vector expressing antisense nucleotide to an LRG) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the exemplary methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain all of the following ingredients, or compounds of a similar nature: a binder such as microcrystalline cellulose, gum tragacanth or gelatin, an excipient such as starch or lactose, a disintegrating agent such as alginic acid, Primogel, or corn starch, a lubricant such as magnesium stearate or Stertes; a glidant such as colloidal silicon dioxide; a sweetening agent such as sucrose or saccharin or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc Liposomal suspensions (including liposomes targeted to infected cells with monoclonal antibodies to viral antigens) can also be used as pharmaceutically-acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated, each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., by assessing the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In many instance, compounds which exhibit large therapeutic indices are used. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. In one embodiment, the dosage of such compounds lies within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

### Kits

The invention also encompasses kits for detecting the presence of an LRG product in a biological sample. The detection can be quantitative or qualitative. The kit may include reagents for assessing the expression of an LRG at nucleotide or protein level. In one embodiment, the reagents include an antibody, or a fragment thereof, which can specifically bind an LRG protein. In another embodiment, the kits comprise a polynucleotide probe which can hybridize under stringent or highly stringent conditions to a transcript of an LRG, or the complement thereof. The kit may contain means for determining the amount of the LRG protein or mRNA in the sample, or means for comparing the amount of the LRG protein or mRNA in the sample with a control or standard. The reagents can be packaged in a suitable container. The kits can further include instructions for using the kit to detect LRG proteins or polynucleotides.

The invention further provides kits for assessing the suitability of each of a plurality of compounds for inhibiting SLE/LN in a subject. Such kits include a plurality of compounds to be tested, and a reagent for assessing the expression of an LRG (e.g., an antibody specific to the corresponding protein, or a probe or primer specific to the corresponding polynucleotide).

### EXAMPLES

### Example 1

### RNA Isolation and Hibridization to Oligonucleolide Arrays

MRL/MpJ-Fas^{lpr}, MRL/MpJ, B6/MRL-Fas^{lPr}, C57BL6/J, SJL/J, Balb/c, and DBA2/J mice were purchased from Jackson Laboratories (Bar Harbor, Me.). Five month old MRL/MpJ-Fas^{lPr} male mice were received as retired breeders. All other mice were obtained at 6 to 8 weeks of age and aged on site.

Kidneys from both male and female mice were collected and snap frozen for RNA isolation. One half of each kidney (a longitudinal section of the left kidney and a cross section of the right kidney) was harvested from each mouse in the study. Snap frozen mouse kidney tissue was homogenized using homogenizer suspended in RLT buffer plus 2-mercaptoethanol for 30 to 45 seconds. Total RNA was prepared using the Qiagen Midi Kit following the manufacturer's protocol. RNA was suspended in DEPC-treated water and quantified by OD 280.

Gene expression analysis was performed on individual kidney RNA samples harvested from the following mice C57BL/6 female mice at 8 weeks (n=3), 20 weeks (n=3) and 32 weeks (n=3); MRL/MpJ-Fas^{lPr} male at 8 weeks (n=3) and 20 weeks (n=2); MRL/MpJ-Fas^{lPr} female mice at 8 weeks (n=3), 16 weeks and 20 weeks (n=6 combined), MRL/MpJ female mice at 8 weeks (n=3) and 20 weeks (n=3), MRL/MpJ male mice at 8 (n=3) and 24 weeks (n=2), B6/MRL-Fas^{lPr} male at 8 weeks (n=3) and 20 weeks (n=3) B6/MRL-Fas^{lPr} female mice at 8 weeks (n=3) and 20 weeks (n=3). Thus, the total number of individual RNA samples subjected to gene expression analysis using the Affymetrix Genechip arrays was 46, twenty-one of which were harvested from lupus nephritis-free strains and the remainder from mice before, during or after disease onset.

cDNA was synthesized from 5 pg of total RNA from each individual kidney sample using the Superscript Kit (Life Technologies, Rockville, Md.). cDNA was purified using phenol: chloroform:isoamyl alcohol (25:24:1) with a Phage lock gel tube following the Phage lock protocol. Supernatant was collected and cleaned up using ethanol. The sample was resuspended in DEPC-treated water.

In vitro T7 polymerase driven transcription reactions for synthesis and biotin labeling of antisense cRNA, Qiagen RNAeasy spin column purification and cRNA fragmentation were carried out as previously described (Lockhart et al., Nature Biotechnology, 14:1675-80, 1996). Genechip hybridization mixtures containing 15 μg fragmented cRNA, 0.5 mg/ml acetylated BSA, and 0.1 mg/ml herring sperm DNA were prepared in 1X MES buffer in a total volume of 200 μl as per manufacturer's instructions. Reaction mixtures were hybridized for 16 hr at 45° C. to Affymetrix Mu11KsubA and Mu11KsubB oligonucleotide arrays. The hybridization mixtures were removed and the arrays were washed and stained with Streptavidin R-phycoerythrin (Molecular Probes, Eugene, Oreg.) using GeneChip Fluidics Station 400 and scanned with a Hewlett Packard GeneArray Scanner following manufactures instructions. Fluorescent data was collected and converted to gene specific difference average using MicroArray Suite software.

### Example 2

### Calculation of Gene Expression Frequency

An eleven member standard curve mixture, comprised of gene fragments derived from cloned bacterial and bacteriophage sequences, was spiked into each hybridization mixture at concentrations ranging from 0.5 pM to 150 pM representing RNA frequencies of approximately 3.3 to 1,000 parts per million (ppm). The biotinylated standard curve fragments were synthesized by T7-polymerase driven IVT reactions from plasmid-based templates. The spiked biotinylated RNA fragments serve both as an internal standard to assess chip sensitivity and as a standard curve to convert measured fluorescent difference averages from individual genes into RNA frequencies in ppm as described by Hill et al., Genome Biol., 2 Res 0055.1-0055.13 (2001). Gene expression frequencies from each individual mouse kidney were measured and the expression data subjected to statistical analysis. Array images were processed using the Affymetrix MicroArray Suite 4 software as follows. Raw array image data (.dat files) were reduced to probe feature-level intensity summaries (.cel files). Probe intensities for each message were then summarized using the Affymetrix Average Difference algorithm, and the Affymetrix Absolute Decision metric was computed (Absent, Present, or Marginal) for each gene. The Average Difference values were converted to estimates of absolute message abundance (in parts per million) by the scaled frequency method as previously described by Hill et al., supra. Briefly, Average Difference values were globally scaled to make the 2% trimmed mean average difference equal for all arrays. Standard curves from spiked cRNAs in each hybridization were then pooled from all arrays, and fitted by a linear calibration function passing through the origin. The scaled Average Difference values from all arrays were multiplied by the slope of this fitted calibration function to give initial frequency estimates. Frequencies smaller than the estimated sensitivity of each array were then adjusted to the average of the frequency and the sensitivity, in order to eliminate negative readouts. Due to the variation in sensitivity among probe sets for different messages, frequencies should be viewed as estimates, and inter-gene comparisons of frequencies should be interpreted cautiously.

### Example 3

### Selection of Genes in Analysis Set

Genes that were not called present by Affymetrix criteria (described below) in at least 50% of samples from at least one group were eliminated from the set of genes under analysis. The Affymetrix Microarray Suite examines the hybridization intensity data from one experiment (probe array) to calculate a set of absolute metrics. The metrics are used by a decision matrix to determine an Absolute Call for each transcript: Present (P), Absent (A), or Marginal (M). Similarly, in order to avoid conclusions dependent on the lower limits of the standard curves, any gene with average frequency not greater than 9 ppm in at least one group was eliminated from analysis. These operations resulted in a list of 5,285 tiled oligonucleotides representing the set of genes to be surveyed for MRL strain-dependent gene expression differences.

In order to identify gene expression patterns that may contributed to disease initiation, genes with significantly different expression levels in young, pre-symptomatic MRL/MpJ kidneys and kidneys from mice that do not develop LN were selected. Late stage disease samples (from MRL/MpJ-Fas^{lpr} mice four months of age or older) were omitted from this initial screen due to the numerous and profound changes in gene expression related to inflammation, kidney failure and fibrosis observed at this stage of disease. These changes may be consequences of the disease process, and would be expected to obscure differences identified between disease-free and early-stage disease samples.

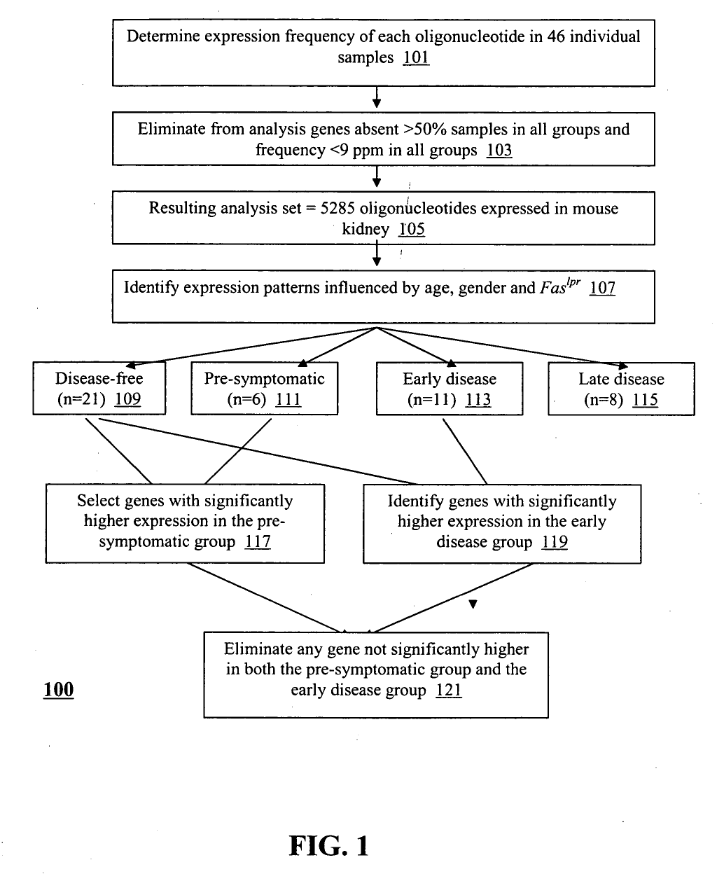

FIG. 1 shows a flow chart describing an exemplary process for selecting LN-related genes that are over-expressed in the pre-symptomatic and early disease groups as compared to the LN-free group. A list of genes with significant expression frequency differences between lupus nephritis negative samples (C57BL/6, C57BL6/Fas^{lPr}) and young (pre-symptomatic) MRL/MpJ kidneys was compiled. Genes on the list had an average fold change (AFC) of greater than 1.5 and a p value of no less than 0.0005 (two-tailed student t-test, unequal variance). Genes that did not also show significant expression level differences (p≦0.0005, AFC>1.5) between the disease-free and early-stage disease samples (consisting of five 20-week or older MRL/MpJ and six 8-week or younger MRL/MpJ-Fas^{lpr} samples) were removed from the list. The step 103 was taken to eliminate genes that had relatively low expression levels. The gene expression patterns influenced by age, gender and Fas^{lPr} were then identified using the resulting gene analysis set of 5285 oligonucleotides in all kidney samples (steps 105-115). Genes with significantly higher expression in the pre-symptomatic group and the early disease group were identified (steps 117 and 119). Finally, only those genes that have significantly higher expression in both groups were selected for further analysis (step 121).

### Example 4

### Flagging of Potential Age, Gender and Fas^{lpr} Dependent Gene Expression Differences

Average fold change (AFC) was obtained by dividing the average frequency of one group by the average of the other group. To identify genes whose expression levels are influenced by gender, the AFC between male and female groups was calculated for each of the six groups of male and female mice listed above. All genes with fold change differences consistent between male and female mice in each group combination were flagged as demonstrating a possible gender influence. Genes with AFC>1.5 between 8 and 32 week old C57BL/6 (disease free) were flagged as “possibly age-influenced.” Genes with AFC>1.5 between C57BL/6 and C57BL/6-Fas^{lpr} were flagged as demonstrating a possible effect of the Fas^{lPr} mutation that did not depend on the disease-prone MRL genetic background. Genes identified through these processes as demonstrating possible gender, age and Fas^{lpr} influences on expression frequency were flagged but retained on the list of genes surveyed for influences related to the MRL genetic background.

Genes associated with age, gender, or Fas^{lpr} influences may still be lupus-related genes that are differentially expressed in lupus-affected or lupus-predisposed tissues relative to disease-free tissues. For diagnostic uses, the reference expression levels of these genes can be determined, for example, by using tissues isolated from reference subjects at the same or comparable age, gender, or Fas^{lPr} background.

### Example 5

### Examples of LRGs

Table 4 shows genes or qualifiers whose hybridization signals on Mu11KsubA or Mu11KsubB oligonucleotide arrays were substantially higher for the pre-symptomatic and early disease samples as compared to the disease-free samples. Accordingly, these genes and qualifiers represent LRGs that are over-expressed in pre-symptomatic and early disease tissues. “Fold change (pre-symptom v. disease free)” represents the ratio of an average frequency of a gene/qualifier in pre-symptomatic samples (e.g., 8-week or younger MRL/MpJ mice) over an average frequency of the same gene/qualifier in disease-free samples (e.g., C57BL/6 or B6/MRL-Fas^{lPr} mice). “Fold Change (early disease v. disease-free)” denotes the ratio of an average frequency of a gene/qualifier in early disease samples (e.g., 8-week or younger MRL/MpJ-Fas^{lpr} or 20-week or older MRL/MpJ mice) over an average frequency of the gene/qualifier in disease-free samples. “Fold Change (late disease v. disease-free)” represents the ratio of an average frequency of a gene/qualifier in late disease samples (e.g., 16-week or older MRL/MpJ-Fas^{lPr} mice) over an average frequency of the gene/qualifier in disease-free samples. The genes or qualifiers in Table 4 do not include those flagged as potentially demonstrating age, gender or Fas^{lPr} dependent expression patterns.

Table 4 also lists the human orthologs that corresponds to each mouse gene or qualifier. These human orthologs can be determined based on Affymetrix annotations, as appreciated by those skilled in the art. Affymetrix ortholog files contain cross-references between probe sets on two different Affymetrix arrays where the reference sequences on which the two probes are based have a significant amount of similarity. The similarity between the reference sequences is determined based on HomoloGene which is a resource of curated and calculated orthologs for genes represented by UniGene or by annotation of genomic sequences (see, for example, the website of the National Center for Biotechnology Information, Bethesda, Md.).

The human orthologs of mouse genes/qualifiers can also be determined by Blast searching human genome databases using the reference sequences or the oligonucleotide probe sequences of the respective qualifiers. The reference sequence or oligonucleotide probe sequences can be readily obtained from the manufacturer of oligonucleotide arrays (e.g., Affymetrix). Human genome databases suitable for Blast search include, but are not limited to, the Entrez nucleotide or genome database at the National Center for Biotechnology Information. Human genes (including putative genes or other transcribable genomic sequences) that significantly align with the reference sequence or the oligonucleotide probe sequence(s) can be identified as the potential human ortholog or homolog of the corresponding mouse qualifier.

For instance, Affymetrix annotation indicates that Mul lKsubA qualifier aa474703_s_at has a human homolog which encodes TIM 14 homolog of yeast TIM14 and is located at chromosome 3q27.2. Blast search of the Entrez human genome database using the reference sequence (tiling sequence) of aa474703_s_at shows that the reference sequence has about 88% sequence identity to LOC390473, a hypothetical gene supported by NM_{—}45261 on chromosome 14. In addition, a fragment of the reference sequence of aa474703_s_at exhibits about 85% sequence identity to a genomic sequence located between CEACAM4 (carcinoembryonic antigen-related cell adhesion molecule 4; LocusID 1089) and CEACAMP3 (carcinoembryonic antigen-related cell adhesion molecule pseudogene 3; LocuslD 1092).

The reference sequence of AA673499_rc_at is 99% identical at the nucleotide level to an AK011097 Mus musculus 13-day enriched liver cDNA:2510042P03:TPR domain-containing protein. The reference sequence of AA689927_s_at is 99% identical to a BC019497 Mus musculus cDNA, Riken cDNA 9430098E02 gene.

Table 5a shows examples of the qualifiers on the Mu11KsubA and Mu11KsubB oligonucleotide arrays that had significantly lower hybridization signals for the pre-symptomatic and early disease samples as compared to disease-free samples. These qualifiers represent genes that are under-expressed in pre-symptomatic and early disease samples relative to lupus-free samples. Genes represented by these qualifier are depicted in Table 5b.

The present invention also contemplates other transcribable human sequences that correspond to or are orthologous to mouse transcripts which are differentially expressed in pre-symptomatic and early-stage lupus-affected samples relative to lupus-free samples. In many instances, these transcribable human sequences have at least 75%, 80%, 85%, 90%, 95%, 98%, 99%, or more sequence identity to the respective mouse transcripts, or the complements thereof.

TABLE 4
Example LRGs that are Over-Expressed in Lupus-Affected Tissues Relative to Disease-Free Tissues

| | | Fold Change | P value | Fold Change | P value | Fold Change | P value |
|---|---|---|---|---|---|---|---|
| Mus musculus | Homo Sapiens | (pre-symptom v. | (pre-symptom v. | (early disease v. | (early disease v. | (late disease v. | (late disease v. |
| Gene/Qualifier | Ortholog | disease free) | disease-free) | disease-free) | disease-free) | disease-free) | disease free) |
| Frg1 | FRG1 | 1.73 | 5.0E−04 | 1.73 | 2.0E−09 | 4.5 | 2.9E−05 |
| Eprs | EPRS | 1.71 | 2.9E−09 | 2.00 | 7.3E−12 | 1.5 | 0.035 |
| Pfn1 | PFN1 | 2.06 | 2.0E−07 | 1.86 | 4.2E−06 | 1.7 | 2.5E−05 |
| Psmd8 | PSMD8 | 2.11 | 2.7E−05 | 1.81 | 1.1E−06 | 1.3 | 8.0E−03 |
| Axin1 | AXIN1 | 3.03 | 4.0E−04 | 2.76 | 5.2E−05 | 2.1 | 0.03 |
| Gnb1 | GNB1 | 1.95 | 1.5E−06 | 1.80 | 3.5E−06 | 1.9 | 5.6E−05 |
| Col4a3 | COL4A3 | 2.64 | 2.6E−04 | 2.22 | 7.5E−05 | 1.9 | 4.4E−08 |
| Hspe1 | HSPE1 | 2.02 | 1.8E−06 | 2.05 | 3.9E−06 | 1.9 | 1.1E−06 |
| Dci | DCI | 1.90 | 2.9E−04 | 2.18 | 3.5E−11 | 1.8 | 6.4E−07 |
| Rcvrn | RCV1 | 1.56 | 5.5E−08 | 1.56 | 2.9E−06 | 1.35 | 6.27E−06 |
| Sfrp1 | SFRP1 | 2.31 | 2.0E−04 | 2.47 | 2.3E−05 | 2.63 | 0.0002 |
| Apom | APOM | 1.79 | 3.1E−04 | 1.64 | 3.9E−04 | 2.19 | 0.068 |
| Kai1 | KAI1 | 2.38 | 5.2E−06 | 2.29 | 7.9E−09 | 2.23 | 3.86E−05 |
| AA689927_s_at | FLJ22709 | 4.28 | 8.6E−05 | 4.90 | 1.5E−06 | | |
| aa177915_at | KIAA0063 | 1.56 | 5.5E−08 | 1.56 | 2.9E−06 | | |
| aa220572_s_at | LOC57019 | 1.94 | 1.3E−06 | 1.88 | 9.3E−06 | | |
| aa474703_s_at | LocusID 131118 | 1.72 | 3.9E−05 | 1.72 | 1.5E−05 | | |
| aa545295_s_at | | 3.40 | 2.5E−04 | 3.37 | 5.0E−10 | | |
| Msa.16987.0_f_at | | 2.11 | 2.7E−05 | 1.81 | 1.1E−06 | | |
| Msa.1705.0_at | GABRB3 | 2.83 | 2.0E−04 | 2.10 | 9.1E−06 | | |
| AA673499_rc_{—} | FLJ30990 | 1.78 | 4.3E−06 | 2.07 | 1.2E−07 | | |
| at | | | | | | | |
| AA472783_at | FLJ38991 | 1.77 | 3.6E−05 | 1.88 | 4.6E−08 | | |
| aa709719_at | CLN6 | 1.60 | 1.8E−04 | 1.53 | 1.0E−07 | | |
| Msa.2399.0_at | DCI | 1.90 | 2.9E−04 | 2.18 | 3.5E−11 | | |

TABLE 5a
Example LRGs that are Under-Expressed in Lupus-Affected Tissues Relative to Disease-Free Tissues

| | Fold Change | P value | Fold Change | P value | Fold Change | P value |
|---|---|---|---|---|---|---|
| Mus musculus | (pre-symptom v. | (pre-symptom v. | (early disease v. | (early disease v. | (late disease v. | (late disease v. |
| Gene/Qualifier | disease free) | disease-free) | disease-free) | disease-free) | disease-free) | disease free) |
| aa466727_s_at | 0.524608501 | 5.98428E−05 | 0.602196 | 3.06576E−05 | 0.540268 | 6.26E−06 |
| aa562768_at | 0.197826087 | 1.33665E−05 | 0.213043 | 1.00476E−05 | 0.121739 | 2.26E−06 |
| aa198618_s_at | 0.589473684 | 1.37891E−06 | 0.65311 | 9.6954E−06 | 0.746053 | 0.000632 |
| aa407822_at | 0.56 | 1.70897E−09 | 0.687273 | 2.51731E−05 | 0.7525 | 0.000764 |
| aa209596_s_at | 0.648809524 | 9.6824E−07 | 0.685065 | 5.15678E−06 | 0.803571 | 0.030387 |
| aa177231_s_at | 0.65915805 | 1.22222E−06 | 0.734591 | 2.24587E−06 | 0.711503 | 4.82E−07 |
| aa250449_s_at | 0.450920245 | 1.36042E−07 | 0.63246 | 9.25536E−06 | 0.756902 | 0.010938 |
| aa607889_at | 0.519138756 | 1.56707E−08 | 0.520661 | 1.71446E−08 | 0.489833 | 3.64E−07 |
| aa289858_s_at | 0.306010929 | 3.2495E−07 | 0.52161 | 8.06798E−05 | 0.645492 | 0.003293 |
| aa408325_rc_s_at | 0.610694184 | 0.000146464 | 0.719939 | 7.50231E−05 | 0.669794 | 8.92E−07 |
| aa277107_s_at | 0.686478455 | 0.000286111 | 0.733284 | 0.000155321 | 0.70013 | 2.58E−05 |
| I42115_s_at | 0.498850575 | 4.96427E−05 | 0.697806 | 7.35745E−05 | 0.724138 | 0.006776 |
| AA237535_s_at | 0.637259503 | 1.98785E−10 | 0.757903 | 7.03074E−07 | 0.631922 | 9.03E−07 |
| AA184872_s_at | 0.15819209 | 4.03569E−14 | 0.399076 | 2.06655E−10 | 0.415254 | 2.24E−07 |
| u37720_f_at | 0.664853556 | 6.64826E−09 | 0.676569 | 7.16556E−08 | 0.724895 | 1.39E−06 |
| aa689125_at | 0.4 | 9.87353E−06 | 0.567273 | 3.08654E−10 | 0.585 | 6.64E−07 |
| aa476184_s_at | 0.324503311 | 4.81241E−11 | 0.59422 | 6.77277E−06 | 0.608444 | 0.002888 |
| aa183627_s_at | 0.554675119 | 1.16862E−09 | 0.559718 | 1.00171E−08 | 0.549128 | 4.48E−08 |
| aa261061_s_at | 0.431404073 | 3.83609E−18 | 0.392868 | 1.01689E−16 | 0.298232 | 4.61E−12 |
| m29881_f_at | 0.11691023 | 5.7402E−08 | 0.352723 | 8.06786E−06 | 2.498956 | 0.014719 |
| AA238219_f_at | 0.407692308 | 3.69198E−13 | 0.472028 | 1.91899E−13 | 0.3375 | 1.02E−14 |
| aa422527_s_at | 0.526483051 | 7.29044E−05 | 0.457049 | 2.2917E−10 | 0.372617 | 2.23E−09 |
| aa689125_g_at | 0.535294118 | 0.000351565 | 0.576471 | 1.43634E−09 | 0.617647 | 1.67E−07 |
| u73200_s_at | 0.616290019 | 2.26269E−05 | 0.636364 | 1.47399E−07 | 0.516596 | 4.74E−11 |
| aa172909_f_at | 0.488372093 | 6.85351E−05 | 0.394027 | 2.32423E−06 | 0.480741 | 5.33E−05 |
| aa408822_rc_s_at | 0.427419355 | 3.88878E−05 | 0.382698 | 4.15819E−09 | 0.33871 | 5.96E−09 |
| d16142_f_at | 0.752683305 | 8.21377E−08 | 0.736924 | 2.70685E−07 | 0.722019 | 0.000124 |
| aa396029_s_at | 0.483164983 | 2.48171E−06 | 0.662075 | 5.08906E−05 | 0.892677 | 0.333131 |
| U59761_s_at | 0.606613455 | 5.64009E−06 | 0.642169 | 7.17506E−09 | 0.667474 | 1.63E−07 |
| aa018016_s_at | 0.2 | 4.96946E−11 | 0.335065 | 1.0996E−09 | 0.364286 | 1.09E−08 |
| aa217493_s_at | 0.343558282 | 2.67279E−07 | 0.339654 | 1.08307E−13 | 0.305982 | 4.62E−11 |
| aa178464_at | 0.715555556 | 0.000404793 | 0.789091 | 0.000433418 | 0.758333 | 7E−05 |
| C77647_rc_at | 0.548192771 | 1.19943E−06 | 0.701533 | 0.000215447 | 0.806476 | 0.050446 |
| m65132_s_at | 0.527932961 | 0.000108643 | 0.725241 | 0.000119675 | 0.76257 | 0.020534 |
| aa120636_s_at | 0.07977208 | 7.85156E−14 | 0.212121 | 5.12728E−13 | 0.201923 | 4.02E−13 |
| I40632_s_at | 0.507246377 | 2.11906E−06 | 0.567194 | 1.82625E−05 | 0.54212 | 2.35E−05 |
| D00926_s_at | 0.713592233 | 0.000236701 | 0.695057 | 1.17067E−05 | 0.603155 | 3.69E−05 |
| aa407794_rc_at | 0.386206897 | 2.45573E−06 | 0.645141 | 5.19105E−05 | 0.57931 | 4.05E−05 |
| aa386606_s_at | 0.547546012 | 5.5361E−07 | 0.650028 | 0.000510386 | 0.539494 | 0.000246 |
| aa710868_at | 0.748091603 | 1.1092E−09 | 0.752949 | 8.44772E−10 | 0.915076 | 0.207197 |
| aa123450_at | 0.565868263 | 1.18718E−06 | 0.74687 | 8.08387E−06 | 0.696856 | 5.84E−05 |
| aa189345_s_at | 0.527108434 | 1.86796E−07 | 0.684283 | 3.3201E−06 | 0.640437 | 9.7E−06 |
| aa175784_s_at | 0.530848329 | 1.11316E−06 | 0.549661 | 6.95076E−09 | 0.695051 | 0.002339 |
| aa170668_s_at | 0.301886792 | 2.55131E−05 | 0.45283 | 0.000206136 | 2.363208 | 0.026526 |
| C78067_rc_at | 0.202312139 | 1.21507E−06 | 0.452444 | 0.000336565 | 0.470376 | 0.000633 |
| aa596794_s_at | 0.450542005 | 2.17246E−10 | 0.513489 | 2.41564E−10 | 0.716717 | 0.071892 |
| aa617621_s_at | 0.680119581 | 1.53675E−09 | 0.7819 | 3.32944E−05 | 0.702354 | 2.39E−07 |
| D50527_f_at | 0.765265923 | 1.58204E−05 | 0.576822 | 3.52814E−06 | 0.658979 | 0.018112 |
| d89076_s_at | 0.240740741 | 3.68613E−12 | 0.40404 | 3.32076E−10 | 0.458333 | 1.88E−07 |
| AF019249_s_at | 0.712962963 | 2.57805E−05 | 0.795455 | 0.000312917 | 1.75 | 0.004574 |
| aa409826_rc_s_at | 0.349493488 | 5.86047E−14 | 0.378503 | 1.01089E−14 | 0.547033 | 3.09E−06 |
| aa204482_s_at | 0.557971014 | 1.69973E−05 | 0.645586 | 6.48639E−08 | 0.98913 | 0.935523 |
| AA060336_at | 0.388888889 | 2.96378E−06 | 0.676136 | 0.000406196 | 0.674479 | 0.003876 |
| aa122805_s_at | 0.147887324 | 7.94241E−16 | 0.203585 | 3.4993E−16 | 0.221831 | 1.01E−15 |
| aa271181_s_at | 0.385 | 3.46746E−08 | 0.534545 | 1.72178E−07 | 0.55125 | 1.28E−06 |
| aa270341_s_at | 0.626315789 | 2.34606E−06 | 0.602871 | 5.23309E−07 | 0.643421 | 0.004666 |
| m59377_s_at | 0.581132075 | 0.000232737 | 0.698799 | 0.000172583 | 0.931132 | 0.618527 |
| aa271360_s_at | 0.69 | 3.6742E−06 | 0.696364 | 2.83724E−16 | 0.6825 | 3.92E−10 |
| af023258_s_at | 0.619856887 | 3.57198E−05 | 0.683038 | 0.000447116 | 0.74195 | 0.00223 |
| aa638759_at | 0.441919192 | 8.4675E−07 | 0.549587 | 5.89466E−07 | 0.662879 | 0.000242 |
| D78255_at | 0.485148515 | 1.80126E−07 | 0.661566 | 0.000267326 | 0.532797 | 4.37E−06 |
| aa028386_at | 0.502217295 | 5.9572E−11 | 0.560572 | 8.22179E−15 | 0.439856 | 1.56E−08 |
| aa271471_s_at | 0.720947631 | 6.74804E−06 | 0.775062 | 9.98855E−05 | 0.687344 | 3.06E−08 |
| aa222947_at | 0.158647141 | 5.16259E−14 | 0.197033 | 7.50847E−15 | 0.219665 | 3.03E−14 |
| aa574478_r_at | 0.267857143 | 8.67266E−10 | 0.457792 | 1.13311E−07 | 0.495536 | 7.01E−07 |
| AA276848_at | 0.66293279 | 0.000156602 | 0.563784 | 4.07868E−09 | 0.545316 | 0.000487 |
| aa414419_s_at | 0.398373984 | 1.54556E−09 | 0.698448 | 0.000403674 | 0.661585 | 0.000359 |
| aa198971_s_at | 0.176780077 | 9.27948E−18 | 0.196869 | 1.14858E−18 | 0.182304 | 3.24E−18 |
| aa066638_s_at | 0.737780041 | 6.75172E−07 | 0.704731 | 3.73292E−09 | 0.539969 | 2.8E−10 |
| aa212803_at | 0.454410307 | 5.74919E−05 | 0.399225 | 3.66647E−13 | 0.379832 | 6.34E−16 |
| U44731_s_at | 0.101694915 | 2.00846E−05 | 0.198767 | 7.9279E−05 | 0.661017 | 0.200896 |
| Msa.409.0_f_at | 0.599331104 | 0.00030862 | 0.57592 | 3.02652E−09 | 0.477592 | 4.08E−10 |
| x00246_f_at | 0.330525778 | 8.93599E−08 | 0.588612 | 0.000327942 | 2.130551 | 0.002183 |
| Msa.24975.0_s_at | 0.307017544 | 1.52793E−06 | 0.703349 | 0.000164249 | 0.667763 | 0.001387 |
| Msa.1292.0_at | 0.612662942 | 0.000309402 | 0.618588 | 9.80731E−05 | 0.518156 | 6.88E−07 |
| W08454_s_at | 0.094230769 | 4.11876E−08 | 0.337762 | 5.29233E−06 | 0.323077 | 3.8E−06 |
| X78709_s_at | 0.685527748 | 6.27352E−08 | 0.710456 | 2.0524E−06 | 0.556991 | 7.05E−09 |
| X54511_f_at | 0.068245125 | 1.09134E−06 | 0.202076 | 9.08382E−06 | 0.241295 | 1.82E−05 |
| w11020_g_at | 0.758426966 | 7.92627E−05 | 0.582227 | 9.49616E−09 | 0.526685 | 2.47E−07 |
| Msa.2906.0_f_at | 0.629766861 | 1.29541E−05 | 0.68988 | 0.000240768 | 0.679746 | 0.00028 |
| x75129_s_at | 0.301724138 | 9.85426E−12 | 0.352665 | 1.3525E−12 | 0.788793 | 0.16634 |
| Msa.6658.0_f_at | 0.609311741 | 1.02916E−06 | 0.564225 | 1.02385E−07 | 0.510121 | 3.52E−05 |
| Msa.34974.0_s_at | 0.281021898 | 3.09018E−08 | 0.543464 | 2.10048E−05 | 0.593978 | 0.000956 |
| Msa.22407.0_s_at | 0.392 | 4.1843E−09 | 0.672 | 1.36783E−06 | 0.63 | 5.21E−05 |
| Msa.35779.0_s_at | 0.653545545 | 1.25771E−05 | 0.646492 | 1.61599E−05 | 0.311106 | 1.75E−08 |
| Msa.4414.0_f_at | 0.47804878 | 2.37512E−07 | 0.577384 | 1.32659E−05 | 0.537805 | 8.57E−05 |
| Msa.3346.0_s_at | 0.706698565 | 2.37997E−06 | 0.728926 | 0.000134707 | 0.567703 | 6.66E−07 |
| w11020_at | 0.648876404 | 0.000111547 | 0.525536 | 1.33463E−06 | 0.626756 | 0.002596 |
| Msa.21579.0_s_at | 0.516666667 | 0.000240218 | 0.481818 | 3.22746E−08 | 0.40625 | 2.13E−08 |
| x04648_s_at | 0.720806794 | 1.7684E−05 | 0.741749 | 5.19421E−05 | 0.863854 | 0.097927 |
| w29651_s_at | 0.674390244 | 1.53487E−05 | 0.58204 | 2.93929E−08 | 0.428963 | 9.71E−11 |
| Msa.3906.0_f_at | 0.412921348 | 0.000280886 | 0.514811 | 0.000278601 | 1.150281 | 0.614585 |
| ET61037_f_at | 0.663553584 | 3.77121E−06 | 0.57344 | 2.00255E−07 | 0.626678 | 0.001914 |
| x16670_f_at | 0.582205029 | 0.000401018 | 0.498505 | 2.24608E−07 | 0.453578 | 6.05E−08 |
| Msa.34568.0_f_at | 0.750349162 | 0.000266617 | 0.654584 | 8.94769E−06 | 0.428946 | 6.33E−08 |
| Msa.19552.0_s_at | 0.087332054 | 6.85921E−05 | 0.073286 | 4.55039E−05 | 0.075576 | 4.68E−05 |
| x04120_f_at | 0.448887837 | 2.38112E−05 | 0.464398 | 2.6399E−06 | 0.475805 | 4.62E−06 |
| X95280_s_at | 0.551971326 | 2.14882E−06 | 0.670577 | 0.000241576 | 0.686828 | 0.008014 |
| x62772_f_at | 0.396907216 | 0.000328691 | 0.511715 | 6.96167E−06 | 0.568299 | 0.000114 |
| Z48043_s_at | 0.615879828 | 1.63436E−05 | 0.639095 | 3.10033E−05 | 0.585837 | 1.82E−05 |

TABLE 5b
Example LRGs that are Under-Expressed in Lupus-Affected Tissues
Relative to Disease-Free Tissues

| Mus musculus | |
|---|---|
| Gene/Qualifier | Gene Name |
| aa466727_s_at | A kinase (PRKA) anchor protein 1 |
| aa562768_at | glioblastoma amplified sequence |
| aa198618_s_at | Mus musculus |
| aa407822_at | RIKEN cDNA 5730494N06 gene |
| aa209596_s_at | translocase of inner mitochondrial membrane 13 homolog a (yeast) |
| aa177231_s_at | RIKEN cDNA 1700051C09 gene |
| aa250449_s_at | RIKEN cDNA 2310016E22 gene |
| aa607889_at | AKAP8 |
| aa289858_s_at | RIKEN cDNA C730049F20 gene |
| aa408325_rc_s_at | 2010300G19RIK |
| aa277107_s_at | sarcosine dehydrogenase |
| l42115_s_at | solute carrier family 1 |
| AA237535_s_at | propionyl Coenzyme A carboxylase |
| AA184872_s_at | RIKEN cDNA 1110023P21 gene |
| u37720_f_at | CDC42 |
| aa689125_at | ZFP277 |
| aa476184_s_at | RIKEN cDNA D530020C15 gene |
| aa183627_s_at | mt27g07.r1 Soares mouse 3NbMS Mus musculus cDNA clone |
| | 622332 5′ |
| aa261061_s_at | RIKEN cDNA 1810010A06 gene |
| m29881_f_at | H2-Q7 |
| AA238219_f_at | solute carrier family 2 (facilitated glucose transporter) |
| aa422527_s_at | RIKEN cDNA 5730591C18 gene |
| aa689125_g_at | ZFP277 |
| u73200_s_at | RHOIP3-PENDING |
| aa172909_f_at | ms20h07.r1 Stratagene mouse skin (#937313) Mus musculus cDNA |
| | clone 607549 5′ similar to gb:M10062 Mouse IgE-binding factor |
| | mRNA; complete cds (MOUSE); |
| aa408822_rc_s_at | EST03349 Mouse 7.5 dpc embryo ectoplacental cone cDNA library |
| | Mus musculus cDNA clone C0033H08 3′ |
| d16142_f_at | peroxiredoxin 1 |
| aa396029_s_at | signal transducer and activator of transcription 3 |
| U59761 _s_at | complete cds. |
| aa018016_s_at | mh45c07.r1 Soares mouse placenta 4NbMP13.5 14.5 Mus musculus |
| | cDNA clone 445452 5′ |
| aa217493_s_at | dynactin 6 |
| aa178464_at | RIKEN cDNA 2210408F11 gene |
| C77647_rc_at | C77647 |
| m65132_s_at | MUC1 |
| aa120636_s_at | serine/threonine kinase 25 (yeast) |
| l40632_s_at | ANK3 |
| D00926_s_at | Mouse mRNA for transcription factor S-II-releated protein |
| aa407794_rc_at | DNA segment |
| aa386606_s_at | CDK5 regulatory subunit associated protein 3 |
| aa710868_at | 4632419I22RIK |
| aa123450_at | RIKEN cDNA 4921505F14 gene |
| aa189345_s_at | caspase 9 |
| aa175784_s_at | viral hemorrhagic septicemia virus(VHSV) induced gene 1 |
| aa170668_s_at | Mus musculus diabetic nephropathy-related gene 1 mRNA |
| C78067_rc_at | BUB3 |
| aa596794_s_at | F2R |
| aa617621_s_at | 2410016C14RIK |
| D50527_f_at | UBC |
| d89076_s_at | TTR |
| AF019249_s_at | NMI |
| aa409826_rc_s_at | D4WSU27E |
| aa204482_s_at | CD97 antigen |
| AA060336_at | RIKEN cDNA 2900086B20 gene |
| aa122805_s_at | ARP3 actin-related protein 3 homolog (yeast) |
| aa271181_s_at | SnRNP assembly defective 1 homolog |
| aa270341 _s_at | hypothetical protein MGC30714 |
| m59377_s_at | TNFRSF1A |
| aa271360_s_at | RIKEN cDNA 1110031B06 gene |
| af023258_s_at | SLC27A1 |
| aa638759_at | AA536743 |
| D78255_at | RP9H |
| aa028386_at | ATP-binding cassette |
| aa271471_s_at | ATP citrate lyase |
| aa222947_at | SLC15A2 |
| aa574478_r_at | 5730414C17RIK |
| AA276848_at | chloride channel 4-2 |
| aa414419_s_at | DNA segment |
| aa198971_s_at | solute carrier family 25 (mitochondrial carrier; peroxisomal |
| | membrane protein) |
| aa066638_s_at | RIKEN cDNA 2310005O14 gene |
| aa212803_at | vanin 1 |
| U44731_s_at | GBP3 |
| Msa.409.0_f_at | complete cds |
| x00246_f_at | histocompatibility 2 |
| Msa.24975.0_s_at | STK25 |
| Msa.1292.0_at | WT1 |
| W08454_s_at | TM4SF8-PENDING |
| X78709_s_at | nuclear factor |
| X54511_f_at | capping protein (actin filament) |
| w11020_g_at | 1810045K07RIK |
| Msa.2906.0_f_at | UBC |
| x75129_s_at | XDH |
| Msa.6658.0_f_at | ARL3 |
| Msa.34974.0_s_at | CD97 |
| Msa.22407.0_s_at | 5330434F23RIK |
| Msa.35779.0_s_at | DNASE1 |
| Msa.4414.0_f_at | 1110033J19RIK |
| Msa.3346.0_s_at | LSM4 |
| w11020_at | 1810045K07RIK |
| Msa.21579.0_s_at | ABCC2 |
| x04648_s_at | Fc receptor |
| w29651_s_at | PLA2G12 |
| Msa.3906.0_f_at | LGALS3 |
| ET61037_f_at | UNK_ET61037 |
| x16670_f_at | UNK_X16670 |
| Msa.34568.0_f_at | HSPCB |
| Msa.19552.0_s_at | UNK_AA013976 |
| x04120_f_at | M.musculus intracisternal A-particle IAP-IL3 genome deleted type |
| | I element inserted 5′ to the interleukin-3 gene. |
| X95280_s_at | G0/G1 switch gene 2 |
| x62772_f_at | apolipoprotein A-II |
| Z48043_s_at | coagulation factor II (thrombin) receptor-like 1 |

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. A method, comprising:
detecting an expression profile of at least one gene in a biological sample of a subject; and
comparing said expression profile to a reference expression profile of said at least one gene to detect or monitor an autoimmune disease in said subject, wherein said at least one gene is differentially expressed in pre-symptomatic lupus-affected or -predisposed tissues as compared to disease-free tissues.

2. The method of claim 1, wherein said at least one gene is differentially expressed in early-stage lupus-affected tissues as compared to said disease-free tissues.

3. The method of claim 2, wherein said at least one gene is over-expressed in both said pre-symptomatic tissues and early-stage lupus-affected tissues as compared to said disease-free tissues.

4. The method of claim 3, wherein said at least one gene includes one or more genes selected from Table 1.

5. The method of claim 2, wherein said at least one gene includes one or more genes selected from Table 5b.

6. The method of claim 2, wherein said subject is a human.

7. The method of claim 6, wherein said autoimmune disease is lupus nephritis (LN) or systemic lupus erythematosus (SLE).

8. The method of claim 2, wherein said expression profile and said reference expression profile are determined by RT-PCR or immunoassays.

9. The method of claim 2, wherein said pre-symptomatic, early-stage lupus-affected, and disease-free tissues are human kidney tissues.

10. A pharmaceutical composition comprising a pharmaceutically-acceptable carrier and at least one active component selected from the group consisting of:
a polypeptide encoded by a gene which is differentially expressed in pre-symptomatic lupus-affected or -predisposed tissues as compared to disease-free tissues;
a variant of said polypeptide; and
a polynucleotide encoding said polypeptide or said variant.

11. The pharmaceutical composition of claim 10, wherein said pharmaceutical composition is a vaccine formulation capable of eliciting an immune response against a lupus-affected or lupus-predisposed human cell or a component thereof, and wherein said gene is selected from Table 1.

12. A method comprising administering a therapeutically or prophylactically effective amount of said pharmaceutical composition of claim 10 to a subject in need thereof.

13. A pharmaceutical composition comprising a pharmaceutically-acceptable carrier and at least one active component selected from the group consisting of:
an agent capable of modulating the expression of a gene which is differentially expressed in pre-symptomatic lupus-affected or -predisposed tissues relative to disease-free tissues;
an agent capable of binding to, or modulating a biological activity of, a polypeptide encoded by said gene; and
a T cell activated by said polypeptide.

14. The pharmaceutical composition of claim 13, wherein said active component is selected from the group consisting of:
a polynucleotide comprising or encoding an RNA that is capable of inhibiting or decreasing the expression of said gene by RNA interference or an antisense mechanism;
an antibody specific for said polypeptide; and
an inhibitor of the biological activity of said polypeptide,
wherein said gene is over-expressed in said pre-symptomatic tissues relative to said disease-free tissues.

15. The pharmaceutical composition of claim 14, wherein said gene is selected from Table 1.

16. A method comprising administering a therapeutically or prophylactically effective amount of said pharmaceutical composition of claim 15 to a human who has or is predisposed to SLE or LN.

17. The pharmaceutical composition according to claim 15, wherein said active component is a polynucleotide comprising or encoding an siRNA directed to a target sequence selected from Table 3.

18. A diagnostic kit comprising:
a polynucleotide capable of hybridizing under stringent or highly stringent conditions to a sequence selected from SEQ ID NOS: 1-29, or the complement thereof; and
an antibody specific for a polypeptide selected from SEQ ID NOS:30-57.

19. A method comprising:
contacting an agent with lupus-affected or lupus-predisposed cells;
comparing expression profiles or protein activities of at least one gene in said cells before and after said contacting to determine if said agent modulates expression or protein activity of said at least one gene,
wherein said at least one gene is differentially expressed in lupus-affected or lupus-predisposed cells as compared to disease-free cells.

20. A method comprising:
administering an agent to a lupus-affected or lupus-predisposed subject;
comparing expression profiles or protein activities of at least one gene in biological samples of the subject before and after said administering to determine if said agent modulates expression or protein activity of said at least one gene in the subject,
wherein said at least one gene is differentially expressed in lupus-affected or lupus-predisposed kidney tissues as compared to disease-free kidney tissues.
